(19) United States
(12) Patent Application Publication
(10) Pub. No.: US 2021/0383903 A1
(43) Pub. Date: Dec. 9, 2021

(54) PROVIDER-CURATED APPLICATIONS FOR ACCESSING PATIENT DATA IN AN EHR SYSTEM

(71) Applicant: Providence St. Joseph Health, Seattle, WA (US)

(72) Inventors: Maulin P Shah, Portland, OR (US); Guilford T Parsons, Seattle, WA (US); Ari A Robicsek, Seattle, WA (US)

(21) Appl. No.: 16/896,889

(22) Filed: Jun. 9, 2020

(51) Int. Cl. G16H 10/60; G16H 50/70; G16H 15/00

(52) U.S. Cl. CPC G16H 10/60; H04L 67/12; G16H 15/00; G16H 50/70

(57) ABSTRACT

A facility accesses patient data in an EHR system. The facility includes a server configured to access the EHR system and store applications and a computing device configured to display EHR data, including patient data. The computing device additionally receives user input specifying patient inclusion criteria and an action and uses the user input to create an application while displaying the patient data. The created application can compare patient data displayed by the computing device to the patient inclusion criteria, determine if the patient data displayed by the computing device satisfies the patient inclusion criteria, and cause the computing device to take the action based on the determination of whether the patient data displayed by the computing device satisfies the patient inclusion criteria.

### BACKGROUND

A variety of software products store data about patients, including current health data, existing and prior conditions, prior procedures, demographic data, scheduled procedures, etc. Such products are often referred to as electronic health records or electronic medical records, and for simplicity are referred to herein as EHRs.

Healthcare providers collect the patient data stored by EHRs from the patient, lab results, their own observations, test results, other facilities, etc. Providers typically access, update, or make changes to EHR data through terminals, PCs, or other client devices that execute software designed to interact with EHR data. EHR data is often transmitted using HL7 and FHIR protocols.

Healthcare providers use the EHR data when making decisions related to the treatment of patients, such as selecting treatment options, prescribing appropriate medicine, diagnosing patients, etc. The data stored in the EHR system aids providers in determining whether a proposed treatment could work, is dangerous, can be improved, etc.

### BRIEF DESCRIPTION OF THE SEVERAL VIEWS OF THE DRAWINGS

FIG. 1 is a block diagram showing a healthcare computer network in which the facility operates in some embodiments.

FIG. 2 is a block diagram showing some of the components typically incorporated in at least some of the app servers used by the facility in some embodiments.

FIG. 3 is a block diagram showing some of the components typically incorporated in at least some of the provider terminals used by the facility in some embodiments.

FIG. 4 is a display diagram showing a sample patient information terminal screen, presented by the facility in some embodiments.

FIG. 5 is a flow diagram showing a process to create an app using templates, performed by the facility in some embodiments.

FIG. 6 is a display diagram showing a sample app template selection screen, presented by the facility in some embodiments.

FIG. 7 is a display diagram showing a sample app template description screen, presented by the facility in some embodiments.

FIG. 8 is a display diagram showing a sample app template criteria screen, presented by the facility in some embodiments.

FIG. 9 is a display diagram showing a sample app template event selection screen, presented by the facility in some embodiments.

FIG. 10 is a flow diagram showing a process to create an app using guided screens, performed by the facility in some embodiments.

FIG. 11 is a display diagram showing a sample app description screen, presented by the facility in some embodiments.

FIG. 12 is a display diagram showing a sample app criteria screen presented by the facility in some embodiments.

FIG. 13 is a display diagram showing a sample app criteria category screen presented by the facility in some embodiments.

FIG. 14 is a display diagram showing a sample app attribute selection screen presented by the facility in some embodiments.

FIG. 15 is a display diagram showing a sample app attribute definition screen presented by the facility in some embodiments.

FIG. 16 is a display diagram showing a sample app additional criteria screen presented by the facility in some embodiments.

FIG. 17 is a display diagram showing a sample app lab result criteria definition screen presented by the facility in some embodiments.

FIG. 18 is a display diagram showing a sample app lab result criteria value definition screen presented by the facility in some embodiments.

FIG. 19 is a display diagram showing a sample app testing screen presented by the facility in some embodiments.

FIG. 20 is a display diagram showing a sample app action definition screen presented by the facility in some embodiments.

FIG. 21 is a display diagram showing a sample app action trigger definition screen presented by the facility in some embodiments.

FIG. 22 is a display diagram showing a sample app beta testing screen presented by the facility in some embodiments.

FIG. 23 is a table diagram showing sample contents of an app definition table used by the facility in some embodiments.

FIG. 24 is a process to subscribe to and use an app used by the facility in some embodiments.

FIG. 25 is a table diagram showing sample contents an app store table used by the facility in some embodiments.

FIG. 26 is a display diagram showing a sample app store screen presented by the facility in some embodiments.

FIG. 27 is a display diagram showing a sample app alert screen used by the facility in some embodiments.

FIG. 28A is a display diagram showing a sample app banner notification screen used by the facility in some embodiments.

FIG. 28B is a display diagram showing a sample app push notification screen, used by the facility in some embodiments.

FIG. 29 is a process to approve apps used by the facility in some embodiments.

FIG. 30 is a table diagram showing sample contents an app deployment table used by the facility in some embodiments.

### DETAILED DESCRIPTION

The inventors have identified important disadvantages of conventional approaches to using EHR data (“patient data”) in making decisions about the treatment of patients. The first relates to the quantity of data available to healthcare providers when making decisions. Patient data can include data aggregated over multiple years or decades, detailing every procedure, diagnosis, condition, etc. that a patient has had. In order to provide effective and efficient treatment, healthcare providers must understand and consider a significant portion of the information about a patient.

While certain conventional tools exist for automating the analysis of patient data to support patient healthcare decisions, these have significant disadvantages. For example, Epic Rule Builder facilitates the creation of guidelines, rules, and alerts which access EHR data to aid physicians when treating patients. These guidelines, rules, and alerts assist providers by giving them guidelines and warnings when the provider takes a specified action such as prescribing medicine, suggesting treatment, scheduling procedures, etc. Providers who have received extensive training in the use of Epic Rule Builder create these guidelines, rules, and alerts.

The Epic Rule Builder, and similar tools, generally require each guideline, rule, and alert be simultaneously applied to all of the parties being treated in a healthcare network, and cannot be limited individual practices, departments, or providers. Additionally, extensive training is required to create guidelines, rules, or alerts in the current systems. Furthermore, a provider must undergo a long process to ensure that every provider in the healthcare network can adopt that guideline, rule, or alert in order for the healthcare network to approve it. Thus, the current systems do not provide healthcare providers with an efficient and easy to use system to create and flexibly apply guidelines, rules, or alerts.

In response to the inventors' recognition of these disadvantages, they have conceived and reduced to practice a software and/or hardware facility for creating and applying guidelines, rules, or alerts (“apps”) based on patient data. The facility democratizes app creation and allows individual providers to quickly and easily create apps, whose application can vary in scope between affecting an individual patient to affecting every patient in a healthcare network. The app then takes an action based on patient data.

In some embodiments, the facility allows providers to create an app which accesses patient data. In some embodiments, the facility retrieves patient data from an EHR system such as systems created by Epic, Cerner, and Vista. In some embodiments, the facility is able to communicate with EHR systems through tools designed to exchange data between EHR systems, such as FHIR or Redox. In some embodiments, the facility is integrated into an existing EHR system. In some embodiments, the facility communicates with the EHR system by using HL7, HTTPS, REST, SOAP, or other communication protocols. In some embodiments, the facility communicates with the EHR system by using the CDA, XML, JSON, or other data formats. In some embodiments, the facility retrieves additional data from external sources such as the Google Health API or any other non-EHR data source. In some embodiments, an app can access a specific patient's data. In some embodiments, an app can access multiple patients' data. In some embodiments, the facility obtains patient data asynchronously. In some embodiments, the facility obtains patient data synchronously.

In some embodiments, the app performs simple logic functions on patient data to determine whether an action should be taken. In some embodiments, machine learning and AI systems analyze recent trends in patient data and suggest existing apps to a provider. In some embodiments, machine learning and AI systems analyze recent trends in patient data and create apps for a provider. In some embodiments, the app takes an action when a previously specified event occurs, such as when a provider interacts with patient data, patient data is received, a certain amount of time has passed, etc.

In some embodiments, the action an app takes is to display a message to the provider. In some embodiments, an app displays a message to the provider when the provider accesses patient data. In some embodiments, an app displays a message to the provider when the provider takes an action such as prescribing a drug, prescribing a treatment, ordering a test, etc. In some embodiments, an app displays a message to the provider when the facility or EHR system has obtained new patient data. In some embodiments, the app displays the message by displaying an alert box on a terminal accessed by the provider. In some embodiments, the app displays a message by transmitting the message directly to the provider, such as through SMS message, email, pager message, etc.

In some embodiments, a provider can publish an app to an “app store,” and thus make the app available to other providers. In some embodiments, the facility allows a provider to change or edit apps obtained from the app store. In some embodiments, the facility integrates the app store into an existing EHR system. In some embodiments, the facility allows the provider to use specific data when creating an app and then generalize the data to cover a wider variety of cases. In some embodiments, the facility generates suggestions to improve an app while the provider creates the app. In some embodiments, the facility generates suggestions to improve an app after the provider has created the app.

In some embodiments, after a provider publishes their app to the app store, other providers can download the app for use in their practice. In some embodiments, the facility allows providers to change or update downloaded apps. In some embodiments, a healthcare organization limits the use of an app to a certain number of people until the app is approved for widespread use. In some embodiments, groups within a healthcare network can subscribe to an app, and thus cause all providers in the department to have the app.

In some embodiments, a provider can provide a limited release of an app in the app store in order to beta test the app. In some embodiments, the provider can invite other providers to beta test the app while the app is in a limited release state. In some embodiments, the facility allows a provider to test an app before deploying it by displaying a sample output of the app based on data available in the EHR system. In some embodiments, the facility allows a provider to create an app by answering a series of questions detailing what information the provider wants to analyze and what the provider wants to do with the information.

By performing in some or all of the ways discussed above, the facility enables providers to create and publish apps quickly and easily, which assists them and other providers in providing accurate, efficient, and safe patient care.

Also, the facility improves the functioning of computer or other hardware, such as by reducing the dynamic display area, processing, storage, and/or data transmission resources needed to perform a certain task, thereby enabling the task to be performed by less capable, capacious, and/or expensive hardware devices, and/or be performed with less latency, and/or preserving more of the conserved resources for use in performing other tasks or additional instances of the same task. As one example, the facility simplifies the process to create apps to be accessible to any provider without the extensive training and overhead previously required, and allows providers to publish and use those apps at as small or large a scale as needed without needing to undergo the extensive testing required to deploy every app across an entire healthcare network.

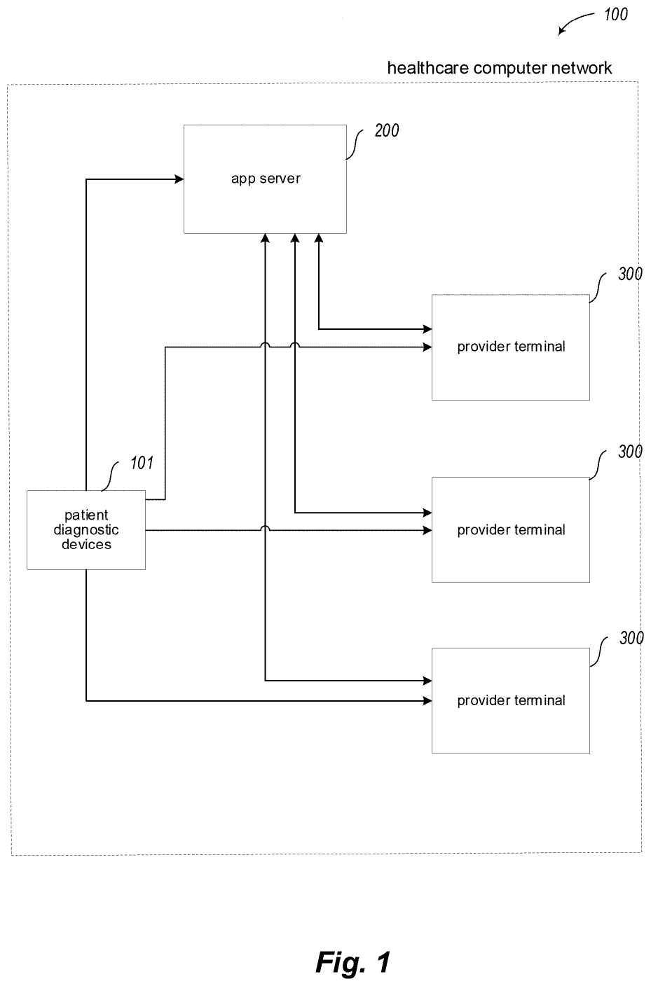

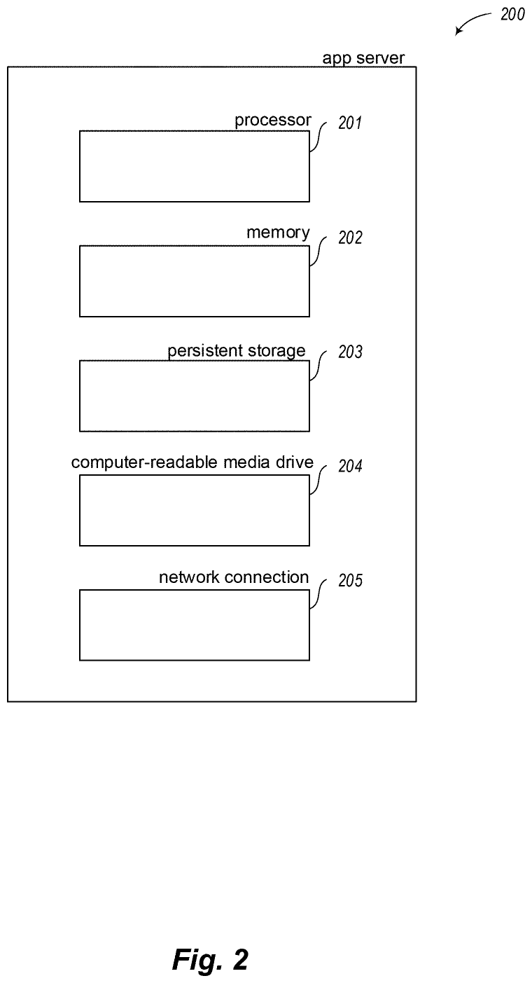

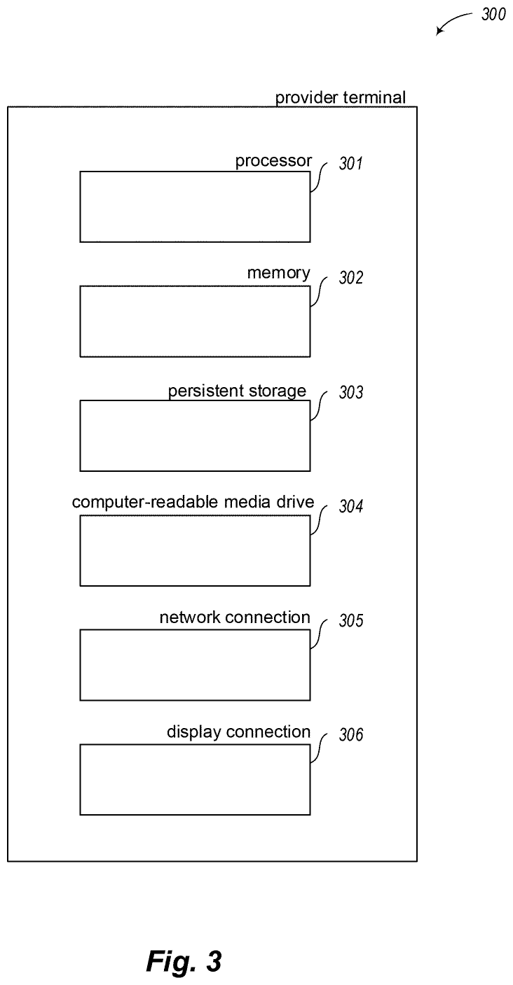

FIG. 1 is a block diagram showing a healthcare computer network 100 in which the facility operates in some embodiments. The healthcare computer network 100 includes patient diagnostic devices 101, an app server 200, and provider terminals 300. The app server 200 stores, transmits, and receives EHR data, and is further described in FIG. 2. The provider terminal 300 provides an interface for healthcare providers to access EHR data and create apps, and is further described in FIG. 3. The patient diagnostic device 101 include EKG machines, lab testing devices, patient monitoring devices, or any other device, tool, or implement used to obtain patient data. In some embodiments, the patient diagnostic devices 101 automatically transmit patient data to the app server 200. In some embodiments, a provider manually enters patient data retrieved from the patient diagnostics devices 101 into the app server 200. In some embodiments, the patient diagnostic devices 101 automatically transmit patient data to one or more of the provider terminals 300. In some embodiments, a provider manually enters data retrieved from the patient diagnostic devices into a provider terminal 300. In some embodiments, the provider terminal 300 retrieves patient data from the app server 200. In some embodiments, the provider terminal 300 transmits patient data to the app server 200. In some embodiments, the app server 200 retrieves patient data from a source external to the healthcare computer network. In some embodiments, the provider terminal 300 retrieves patient data from a source external to the healthcare computer network. In some embodiments, the provider terminal 300 transmits patient data to a computing device external to the healthcare computer network. In some embodiments, the app server 200 transmits patient data to a computing device external to the healthcare computer network.

FIG. 2 is a block diagram showing some of the components typically incorporated in at least some of the app servers 200 used by the facility in some embodiments. In various embodiments, the app server 200 can include a physical server, a cloud server, multiple servers, etc. In various embodiments, the app server 200 includes zero or more of each of the following: a central processing unit (“CPU”) 201 or processor of another type for executing computer programs; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a SD-card, floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may implement devices of various types and configurations, and having various components. In some embodiments, the app server 200 implements an EHR system to access and manage patient data. In some embodiments, the app server 200 retrieves other data from computing devices external to the healthcare computer network of which the app server 200 is a part.

FIG. 3 is a block diagram showing some of the components typically incorporated in at least some of the provider terminals 300 used by the facility in some embodiments. In various embodiments, the provider terminals 300 can include desktop computers, tablet computers, mobile phones, tablet computers, personal digital assistants, laptop computer systems, netbooks, etc. In various embodiments, the provider terminals 300 include zero or more of each of the following: a central processing unit (“CPU”) 301 for executing computer programs; a computer memory 302 for storing programs and data while they are being used, including the facility and associated data, an operating system including a kernel, and device drivers; a persistent storage device 303, such as a hard drive or flash drive for persistently storing programs and data; a computer-readable media drive 304, such as a SD-card, floppy, CD-ROM, or DVD drive, for reading programs and data stored on a computer-readable medium; a network connection 305 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like; and a display connection 306 for displaying visual information or data to a user. While computer systems configured as described above are typically used to support the operation of the facility, those skilled in the art will appreciate that the facility may implement devices of various types and configurations, and having various components.

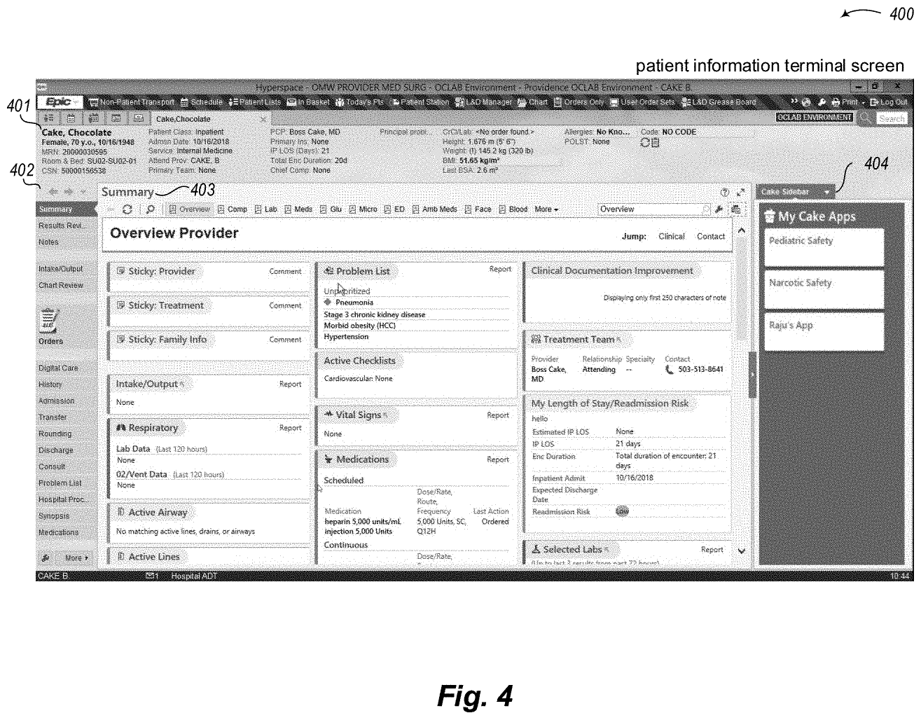

FIG. 4 is a display diagram showing a sample patient information terminal screen for a particular patient being served by one or more particular providers, presented by the facility in some embodiments. The patient information terminal screen 400 includes a patient demographics section 401, a patient summary sidebar 402, a summary tab 403, and an apps sidebar 404. The patient demographics section 401 includes basic demographic information regarding the patient, such as the patient's name, sex, age, and birthday. The patient summary sidebar 402 includes one or more tabs, such as the summary tab 403, each indicating a screen which displays patient data, such as a summary screen, a snapshot screen, an orders screen, a history screen, an allergies screen, etc. The summary tab 403 indicates a summary screen which displays patient data such as the patient's medications, treatment teams, family information, vital signs, etc. The apps sidebar 404 includes zero or more apps which are in use by the provider.

In the example patient information terminal screen 400 depicted by FIG. 4, three of the provider's apps are active for the patient: “Pediatric Safety,” “Narcotic Safety,” and “Raju's App.” In some embodiments, the apps indicated by the app sidebar 404 are all of the apps to which the provider has subscribed. In some embodiments, the facility determines which apps are applicable to the patient based on patient data, and the app sidebar 404 indicates only apps applicable to the patient.

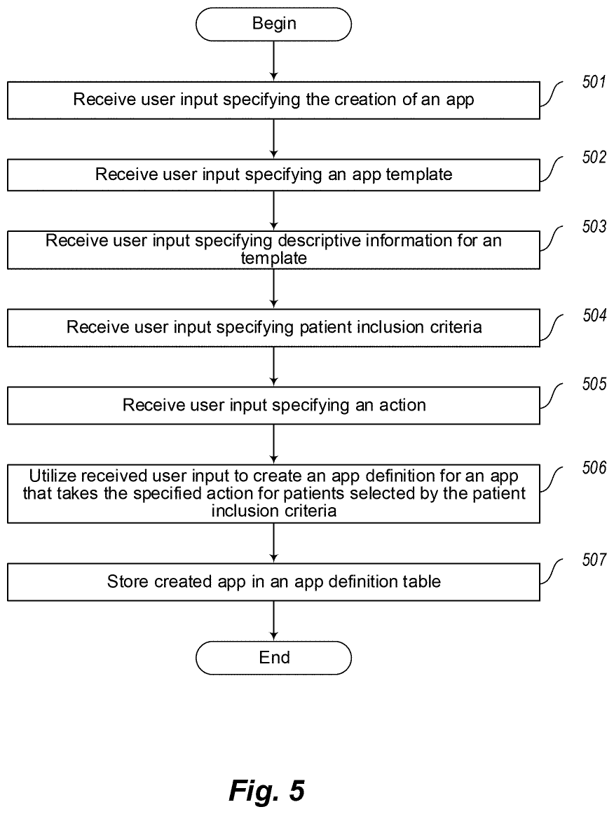

FIG. 5 is a flow diagram showing a process to create an app using templates, performed by the facility in some embodiments. In some embodiments, when performing the process described in FIG. 5, the facility receives user input from a computing device with access to the app server. In some embodiments, when performing the process described in FIG. 5, the facility receives user input at a provider terminal. In some embodiments, when performing the process described in FIG. 5, the facility receives user input at a computing device external to the healthcare computer network. In act 501, the facility receives user input specifying the creation of an app. In act 502, the facility receives user input specifying an app template. In some embodiments, the facility performs act 502 by displaying an app template selection screen to the user.

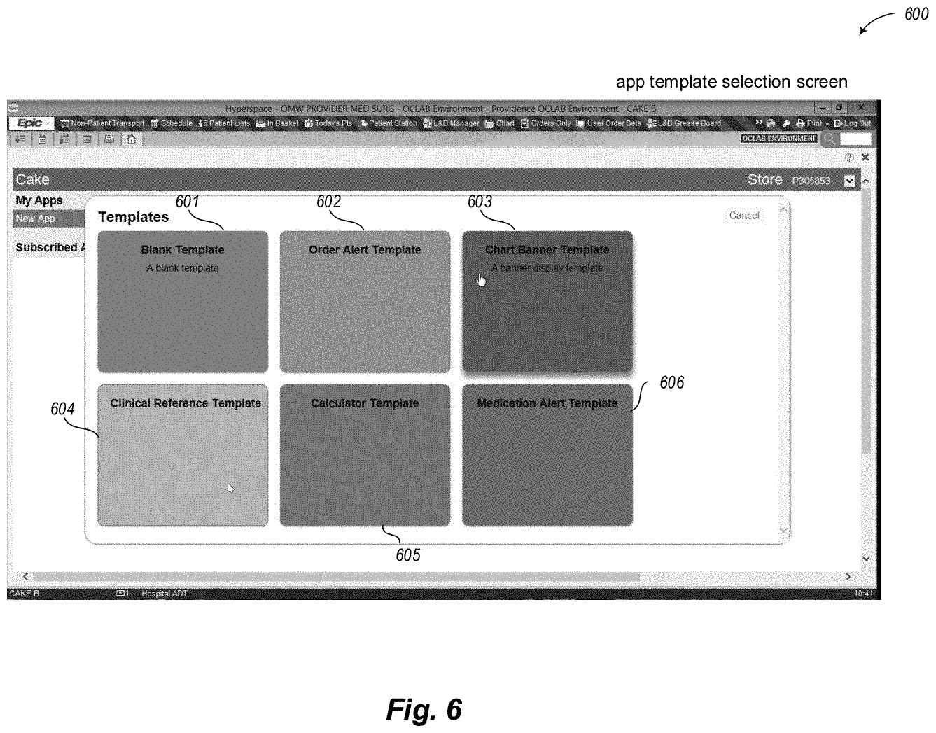

FIG. 6 is a display diagram showing a sample app template selection screen, presented by the facility in some embodiments. The app template selection screen 600 includes a blank template button 601, an order alert template button 602, a chart banner template button 603, a clinical reference template button 604, a calculator template button 605, and a medication alert template 606. The blank template button 601 allows a provider to create a template without any initial settings for the patient inclusion criteria, action, extra information, etc. The order alert template button 602 allows a provider to create a template with initial settings specifying the action as displaying an alert box when a provider enters a new order for a patient. The chart banner template button 603 allows a provider to create a template with initial settings specifying the action as displaying a banner message at the top of a patient chart. The clinical reference template button 604 allows a provider to create a template with initial settings specifying patient inclusion criteria based on lab results. The calculator template button 605 allows a provider to create a template which performs a calculation as part of the patient inclusion criteria, action, or action trigger. The medication alert template button 606 allows a provider to create a template with initial settings specifying an alert to be displayed when a specified type of medication is ordered.

Returning to FIG. 5, in act 503, the facility receives user input specifying descriptive information for an app template. In some embodiments, the facility performs act 503 by displaying an app template description screen.

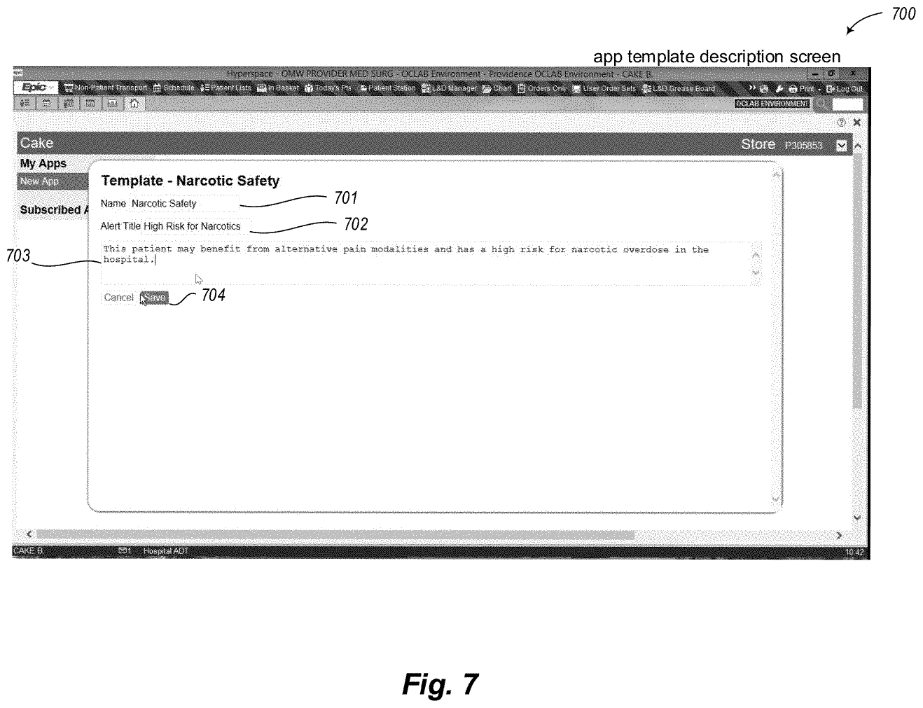

FIG. 7 is a display diagram showing a sample app template description screen, presented by the facility in some embodiments. The app template description screen 700 includes an app name text box 701, an alert title text box 702, an alert content text box 703, and a save button 704. The app name text box 701 allows the provider to input the name of the app they are creating. The alert title text box 702 allows a provider to input a title for the alert displayed by the app. The alert content text box 703 allows the provider to input a message or other content for the alert displayed by the app. When the provider has finished inputting the descriptive information, the provider activates the save button 704.

Returning to FIG. 5, in act 504, the facility receives user input specifying patient inclusion criteria. In some embodiments, the facility performs act 504 by displaying an app template patient criteria screen.

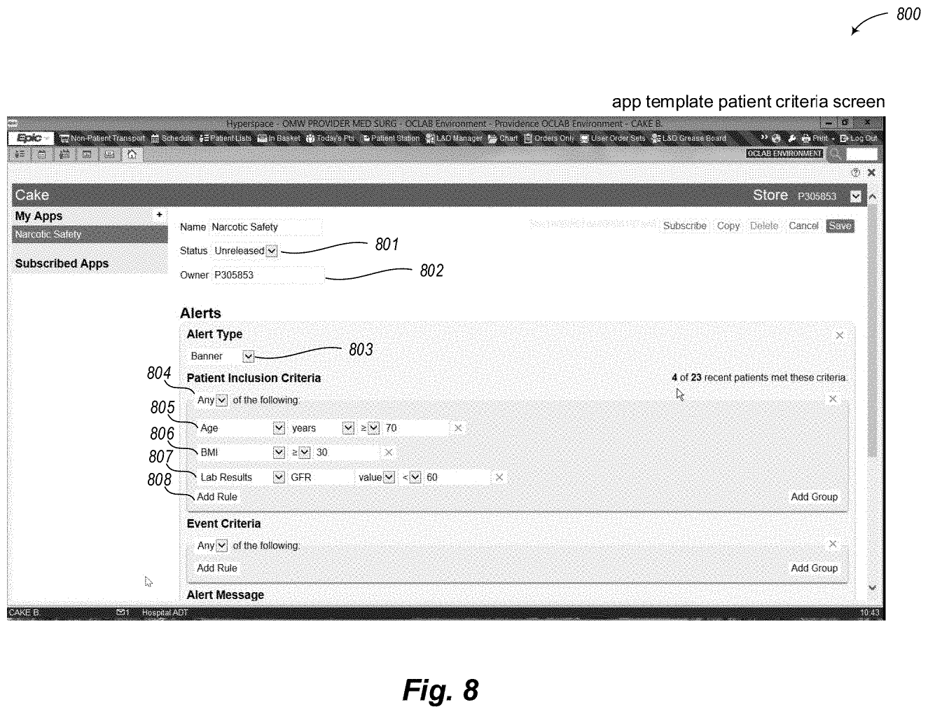

FIG. 8 is a display diagram showing a sample app template criteria screen, presented by the facility in some embodiments. In some embodiments, the app template patient criteria screen 800 includes a status dropdown 801, owner text box 802, alert type dropdown 803, a logical operator dropdown 804, rules 805-807, and an add rule button 808. The status dropdown 801 indicates the current release state of the app, where being released means the app is available to other providers and being unreleased means the app is not yet available to other providers. In some embodiments, the status dropdown 801 includes a beta testing option. The owner text box 802 includes identifying information for the owner of the app. In some embodiments, the owner is the creator of the app. In some embodiments, the owner is not the creator of the app. The alert type dropdown 803 allows the provider to select the type of alert included in the app, such as a banner, a dialog box, a text message, an email, etc.

The logic operator dropdown 804 allows the provider to specify how the facility compares each of the rules to determine if the app applies to the patient. For example, if the logic operator dropdown 804 is set to “Any”—representing a logical OR statement—then if any of the rules are true, the patient will be included. Likewise, if the logic operator dropdown is set to “All”—representing a logical AND statement—all of the rules must be true for the patient to be included. In some embodiments, the logic operator dropdown includes additional values representing other logic operators, such as XOR, NOR, NOT, etc. The rules 805-807 are all rules applied by the facility to determine if the app applies to the patient. After a provider selects a category for one of the rules, such as “Age” for rule 805, the facility adds dropdown boxes to allow the provider to input a requirement for the rule. For example, when rule 805 indicates the “Age” category, the facility prompts the user for a unit, such as years, months, days, etc., a comparison operator, such as “>”, “<”, “=”, etc., and a value. Rule 805 would indicate that a patient whose age in years is greater than or equal to 70 could be included in the app. The categories used by rules 805-807 include any type of patient data, such as age, weight, blood pressure, blood type, recent lab results, patient history, etc. In some embodiments, the rules 805-807 can also include calculations. For example, a rule could include a patient's weight divided by a certain number. The add rule button 808 allows a provider to add another rule, such as rule 806 or rule 807.

Returning to FIG. 5, in act 505, the facility receives user input specifying an action. In some embodiments, the facility performs act 505 by using an app template event selection screen.

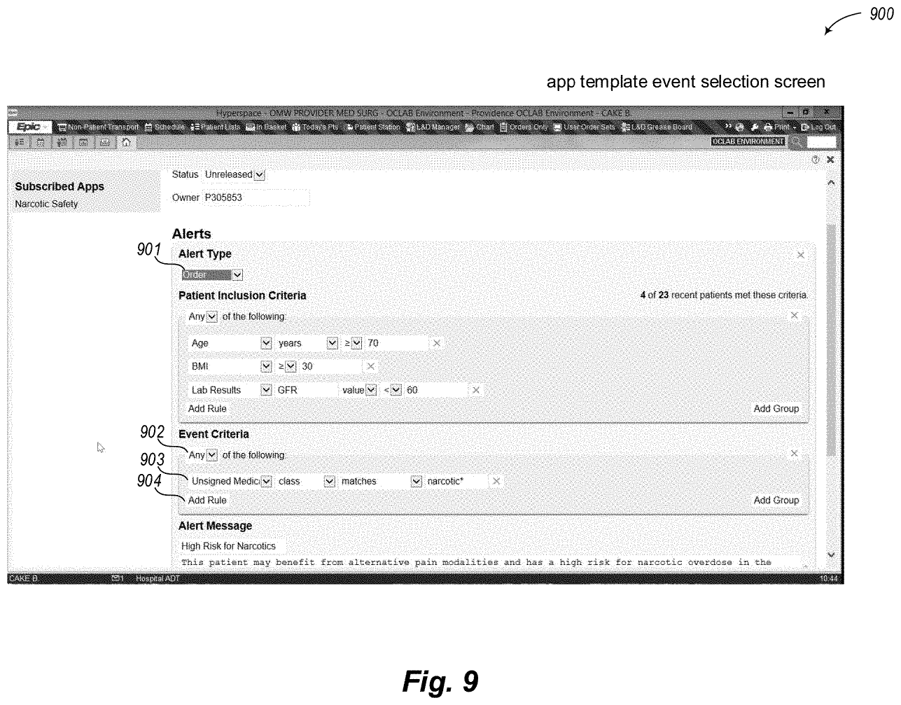

FIG. 9 is a display diagram showing a sample app template event selection screen, presented by the facility in some embodiments. The app template event selection screen 900 includes an alert type dropdown 901, a logic operator dropdown 902, a rule 903, and an add rule button 904. The alert type dropdown 901 operates in a similar fashion to the alert type dropdown 803 shown in FIG. 8. The logic operator dropdown 902 operates in a similar fashion to the logic operator dropdown 804 shown in FIG. 8. The rule 903 operates in a similar fashion to the rules 805-807 shown in FIG. 8. In some embodiments, the rule 903 contains dropdowns which include categories that are not included in the rules 805-807. The add rule button 904 operates in a similar fashion to the add rule button 808 shown in FIG. 8. In some embodiments, the facility uses the input received in the logic operator dropdown 902 and rule 903 to determine when the app should take an action (an “action trigger” or “event trigger”) after the facility has determined that a patient satisfies the patient inclusion criteria described in connection with FIG. 8. For example, if an app's patient inclusion criteria is set to “Age=70” and the app's action trigger is “ordering new medication,” then the facility will take the action when a patient's data indicates they are 70 years old and the provider orders new medication, but will not take the action if the patient is not 70 years old even if the provider has ordered new medication. In some embodiments, the action trigger can include ordering treatment or medication, ordering treatment or medication of a certain type, opening the patient chart, identifying new treatment methods for a patient, such as qualifying for a research study, new drug, newly discovered treatment, etc., receiving new EHR data related to the patient, receiving patient data from a non-EHR data source, etc.

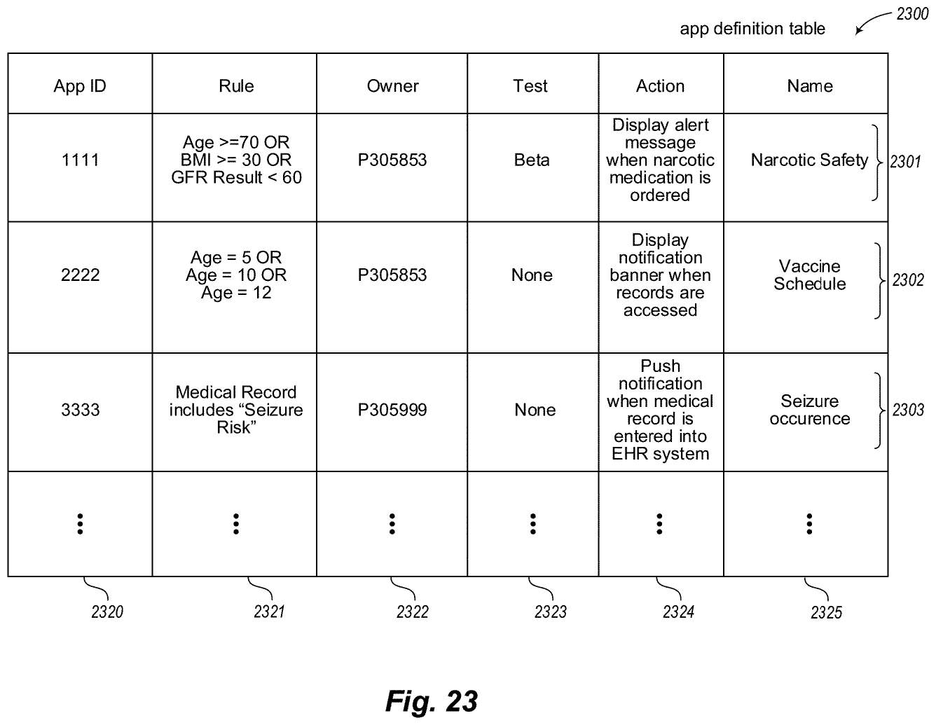

Returning to FIG. 5, in act 506, the facility utilizes the received user input to create an app definition for an app that takes the specified action for patients selected by the patient inclusion criteria. In act 507, the facility stores the created app in an app definition table, such as the app definition table 2300 shown in FIG. 23. After act 507 this process concludes.

Those skilled in the art will appreciate that the acts shown in FIG. 5 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

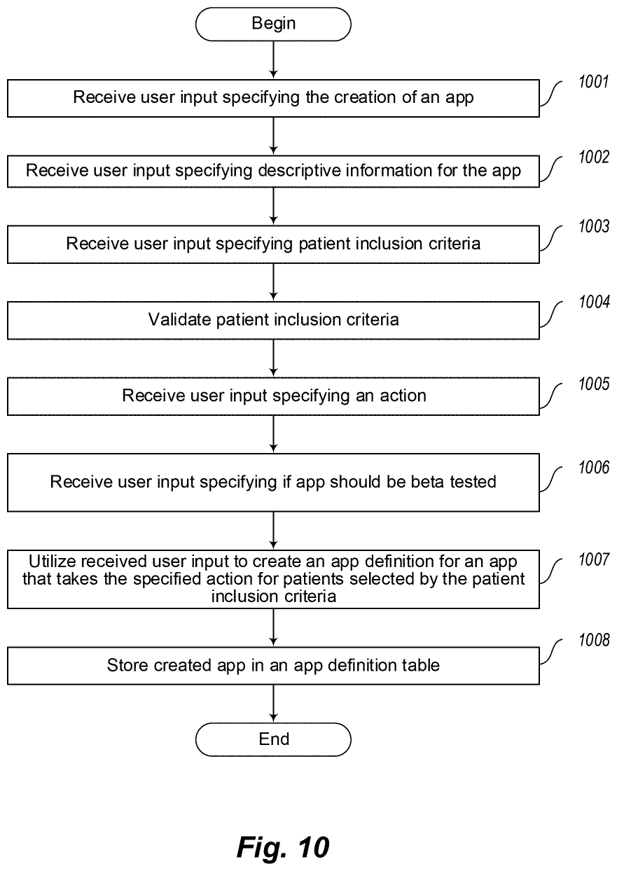

FIG. 10 is a flow diagram showing a process to create an app using guided screens, performed by the facility in some embodiments. In act 1001, the facility receives user input specifying the creation of an app. In act 1002, the facility receives user input specifying descriptive information for the app. In some embodiments, the facility performs act 1002 by using an app description screen.

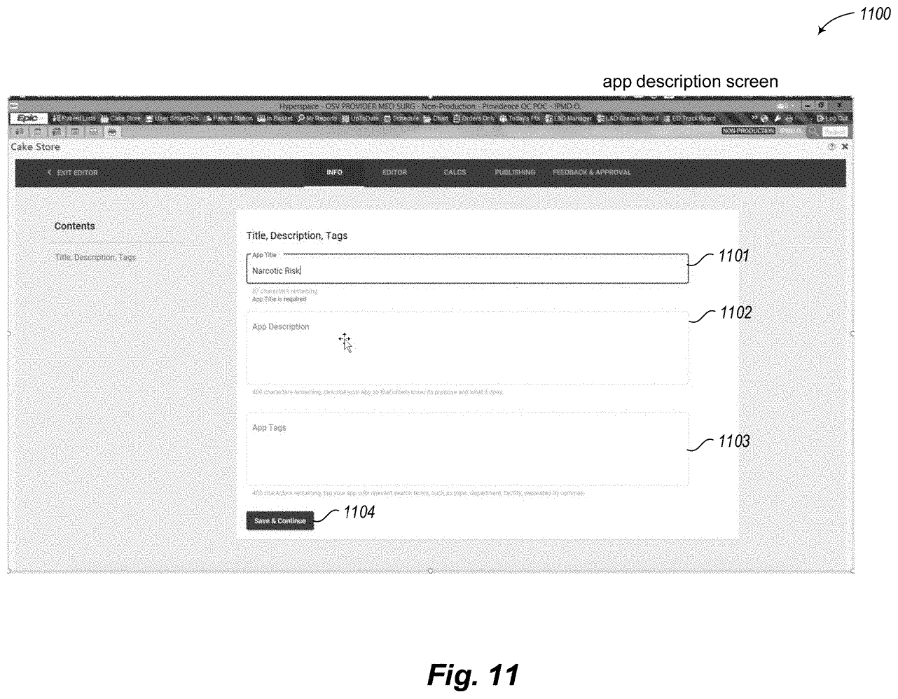

FIG. 11 is a display diagram showing a sample app description screen, presented by the facility in some embodiments. The app description screen 1100 includes a title text box 1101, a description text box 1102, a tags text box 1103, and a save button 1104. The title text box 1101 allows a provider to input a title describing the app. The description text box 1102 allows a provider to input a description of the app. The tags text box 1103 allows a provider to input descriptive tags for the app. In some embodiments, an app store operated by the facility allows other providers to search for apps with similar tags to the tags entered in the tags text box 1103. After the provider has finished inputting information in the app description screen the provider activates the save button 1104.

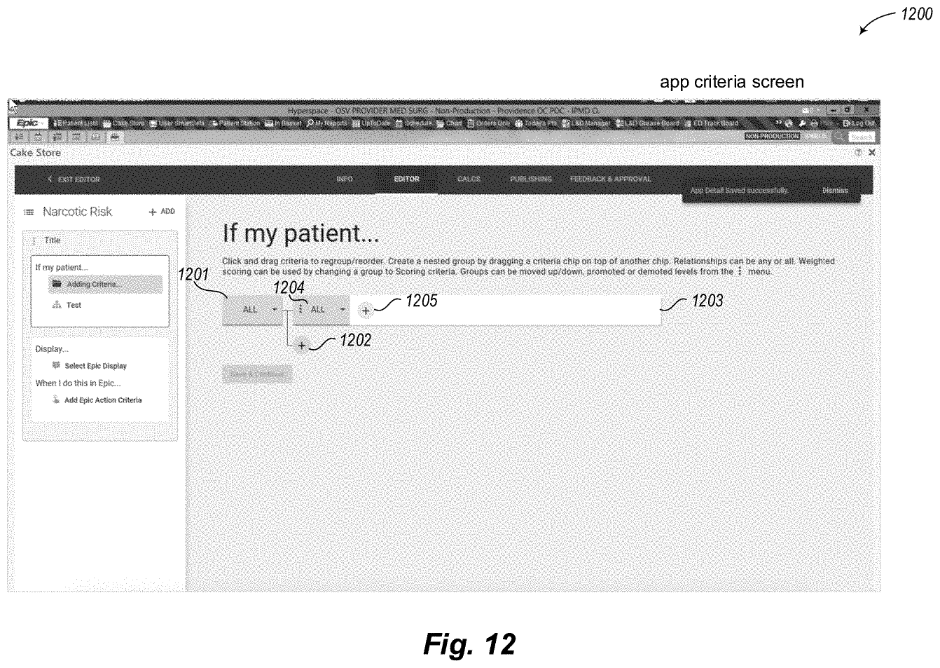

Returning to FIG. 10, in act 1003, the facility receives user input specifying patient inclusion criteria. In some embodiments, the facility performs act 1003 by utilizing the screens shown in FIGS. 12-18.

FIG. 12 is a display diagram showing a sample app criteria screen presented by the facility in some embodiments. The app criteria screen 1200 includes a logic operator selector 1201, an add rule button 1202, and a rule 1203. The logic operator selector 1201 operates similarly to the logic operator dropdown 804. The add rule button 1202 operates similarly to the add rule button 808. The rule 1203 includes its own logic operator selector 1204 and an add rule criteria button 1205. The logic operator selector 1204 operates similarly to the logic operator selector 1201; however, the logic operator selector 1204 only applies to the conditions within the rule 1203, allowing the provider to create rules with nested logic operators, such as, for example, ((Age>=70 AND BMI>30) OR (GFR lab result>30)). The add rule criteria button 1205 allows a provider to add a condition to the rule 1203. In some embodiments, the add rule criteria button 1205 indicates to the facility that an app criteria category screen should be presented to the provider.

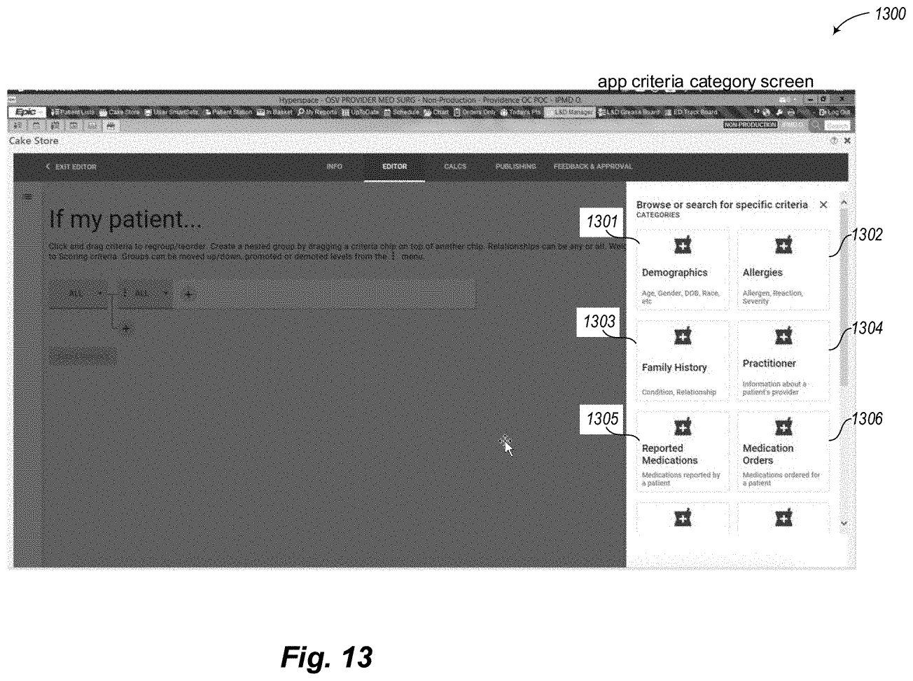

FIG. 13 is a display diagram showing a sample app criteria category screen presented by the facility in some embodiments. The app criteria category screen 1300 includes a demographics category button 1301, an allergies category button 1302, a family history category button 1303, a practitioner button 1304, a reported medications button 1305, and a medication orders button 1306. In some embodiments, the app criteria category screen 1300 includes other buttons accessible by scrolling the page. Each of the buttons 1301-1306, when activated, direct a provider to a separate screen, or screens, designed to obtain information regarding patient inclusion criteria. For example, the demographics button 1301 directs the provider to a screen which assists the provider in specifying patient inclusion criteria related to patient demographics, such as an app attribute selection screen.

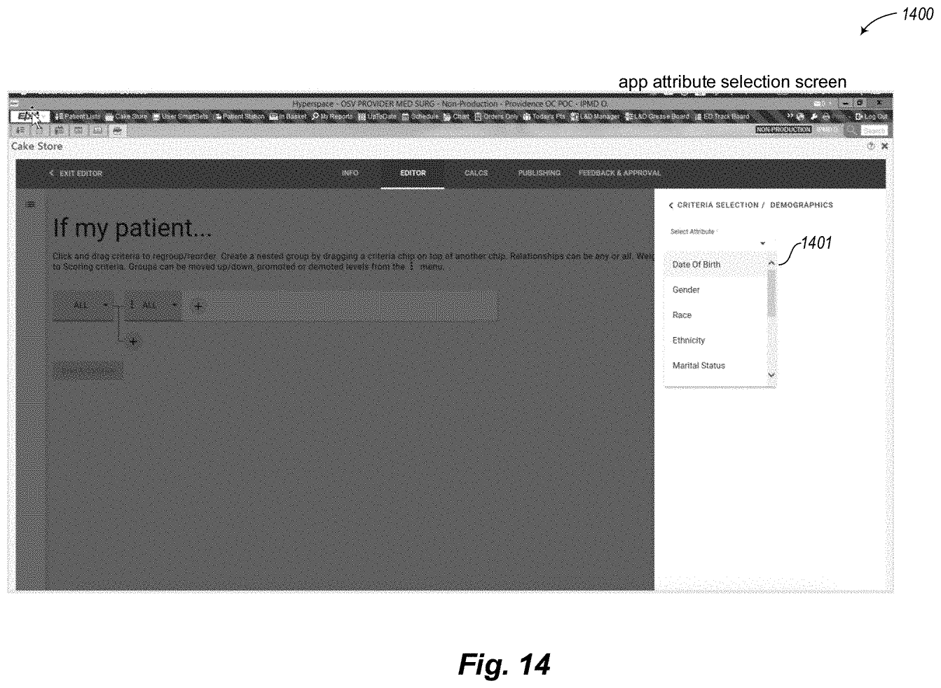

FIG. 14 is a display diagram showing a sample app attribute selection screen presented by the facility in some embodiments. The app attribute selection screen 1400 includes an attribute selection dropdown 1401. The attribute selection dropdown 1401 includes a list of attributes related to the category of criteria selected in the app criteria category screen 1300. For example, here the attribute selection dropdown 1401 includes attributes such as date of birth, gender, race, ethnicity, marital status, etc. because in this example the provider selected the demographics criteria category. In some embodiments, when a provider selects one of the attributes, the facility directs the provider to an app attribute definition screen.

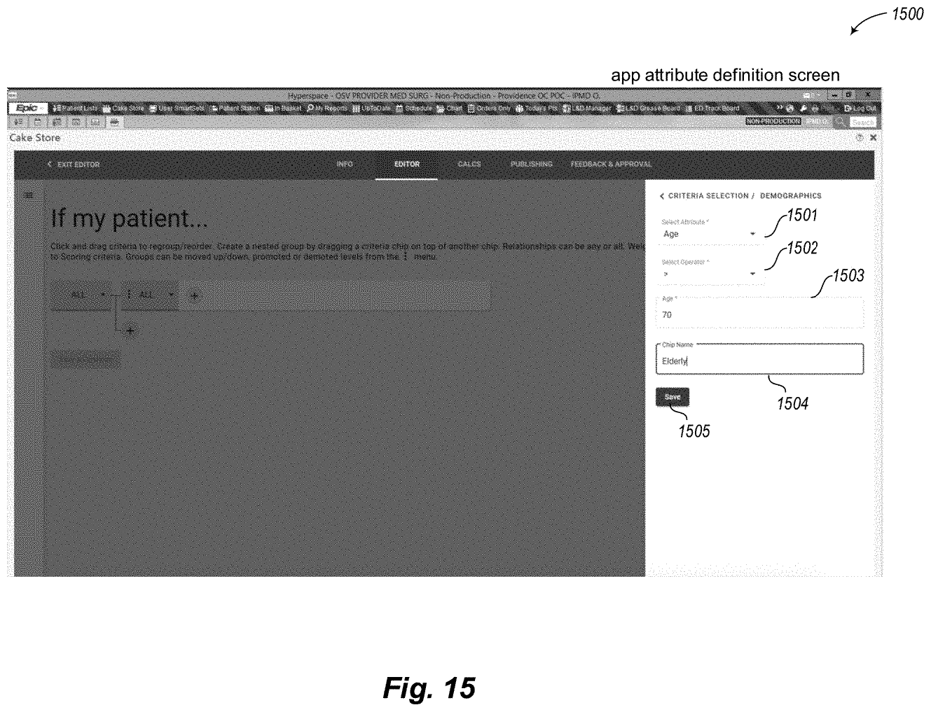

FIG. 15 is a display diagram showing a sample app attribute definition screen presented by the facility in some embodiments. The app attribute definition screen 1500 includes an attribute selection dropdown 1501, a comparison operator dropdown 1502, a value text box 1503, and a name text box 1504. The attribute selection dropdown 1501 operates similarly to the attribute selection dropdown 1401. The comparison operator dropdown 1502 allows a provider to choose a comparison operator such as “>”, “<”, “=”, etc. The value text box 1503 allows a provider to input a value which the facility utilizes to determine if a patient should be included. The name text box 1504 allows a provider to enter text describing the rule criteria. The provider activates the save button 1505 when they have finished inputting data. In some embodiments, the facility directs the user to an app additional criteria screen after the provider has activated the save button 1505.

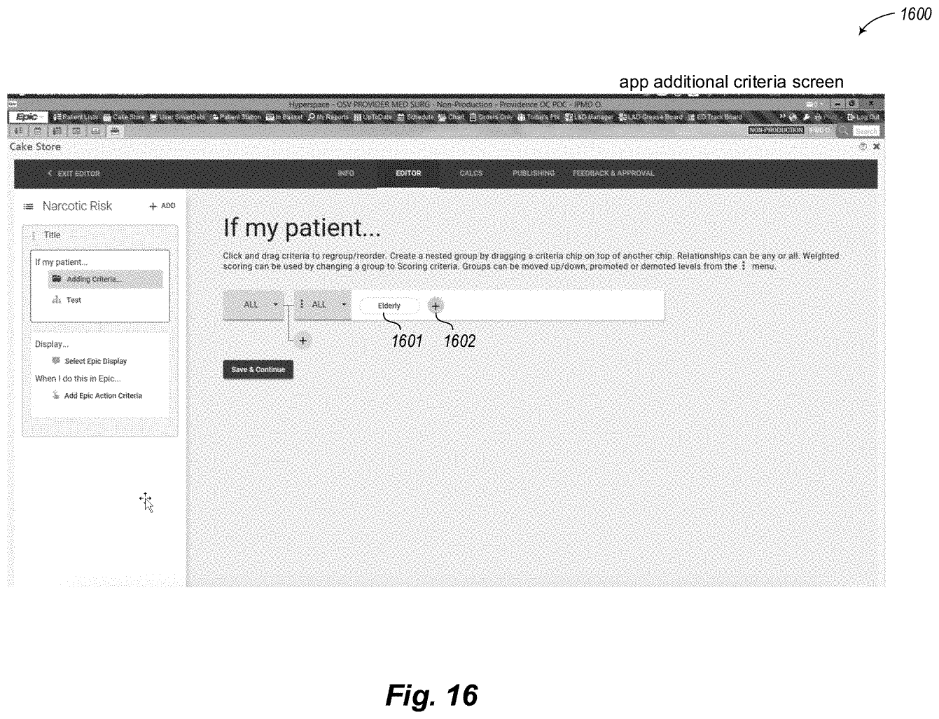

FIG. 16 is a display diagram showing a sample app additional criteria screen presented by the facility in some embodiments. The app additional criteria screen 1600 includes a first rule criteria 1601 and an additional rule criteria button 1602. The first rule criteria 1601 displays the name entered in the name text box 1504, and represents a rule which the provider had previously defined. The additional rule criteria button 1602 allows a provider to input data indicating additional rule criteria for the rule by indicating to the facility to display the app criteria category screen 1300 described in FIG. 13. In some embodiments, the app criteria category screen 1300 includes a lab results button, and the facility directs the provider to an app lab result criteria definition screen when the lab results button is activated.

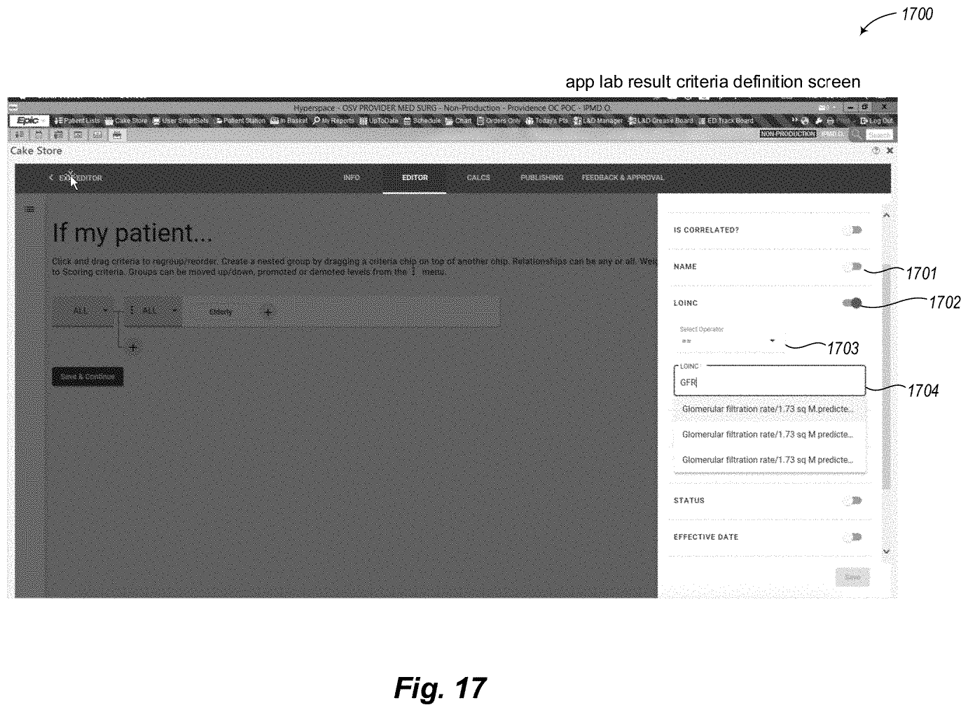

FIG. 17 is a display diagram showing a sample app lab result criteria definition screen presented by the facility in some embodiments. The app lab result criteria definition screen 1700 includes a name option 1701, and a LOINC option 1702. In some embodiments, the app lab result criteria definition screen 1700 includes more options which allow the provider to input data defining the rule criteria. Activating the name option 1701 allows the provider to input text describing the rule criteria. Activating the LOINC option 1702 causes the facility to display the operator dropdown 1703 and the LOINC code textbox 1704. The operator dropdown 1703 operates similarly to the operator dropdown 1502 described in FIG. 15. The LOINC code text box 1704 allows a provider to enter a code identifying a health measurement, observation, health documents, etc., such as a LOINC code. In some embodiments, when a provider enters a code into the LOINC code text box 1704 the facility directs the provider to an app lab result criteria value definition screen.

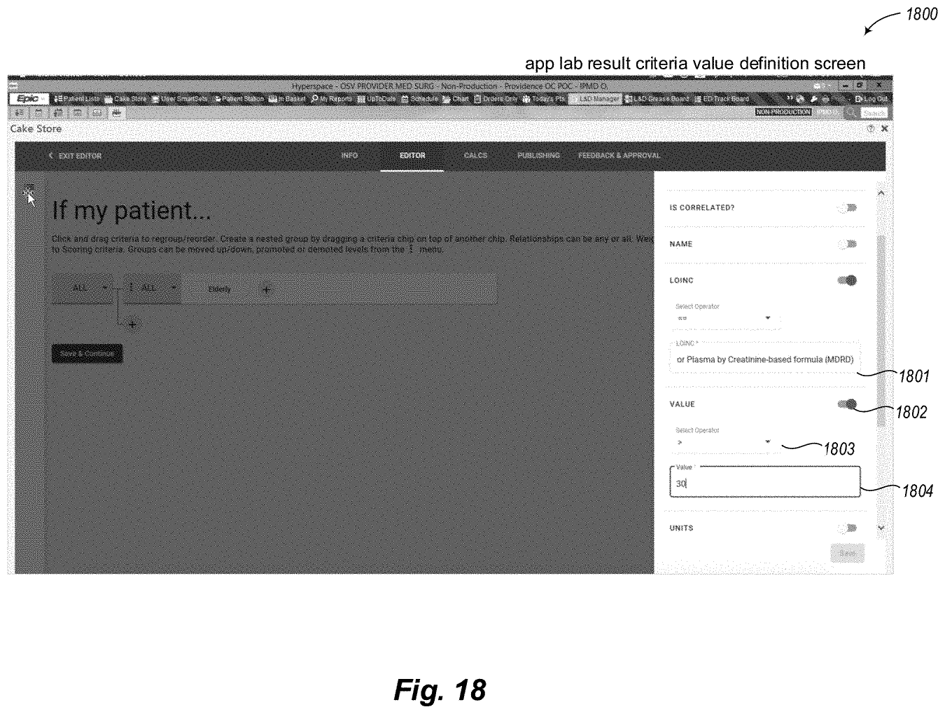

FIG. 18 is a display diagram showing a sample app lab result criteria value definition screen presented by the facility in some embodiments. The app lab result criteria value definition screen 1800 includes a LOINC code text box 1801 and a value option 1802. The LOINC code text box 1801 operates similarly to the LOINC code text box 1704. Activating the value option 1802 causes the facility to display an operator dropdown 1803, and a value text box 1804. The operator dropdown 1803 operates similarly to the operator dropdown 1502 described in FIG. 15. The value text box 1804 allows a provider to input a value which the facility utilizes to determine if a patient should be included.

Returning to FIG. 10, in act 1004, the facility validates the patient inclusion criteria. In some embodiments, the facility utilizes an app testing screen to validate the patient inclusion criteria.

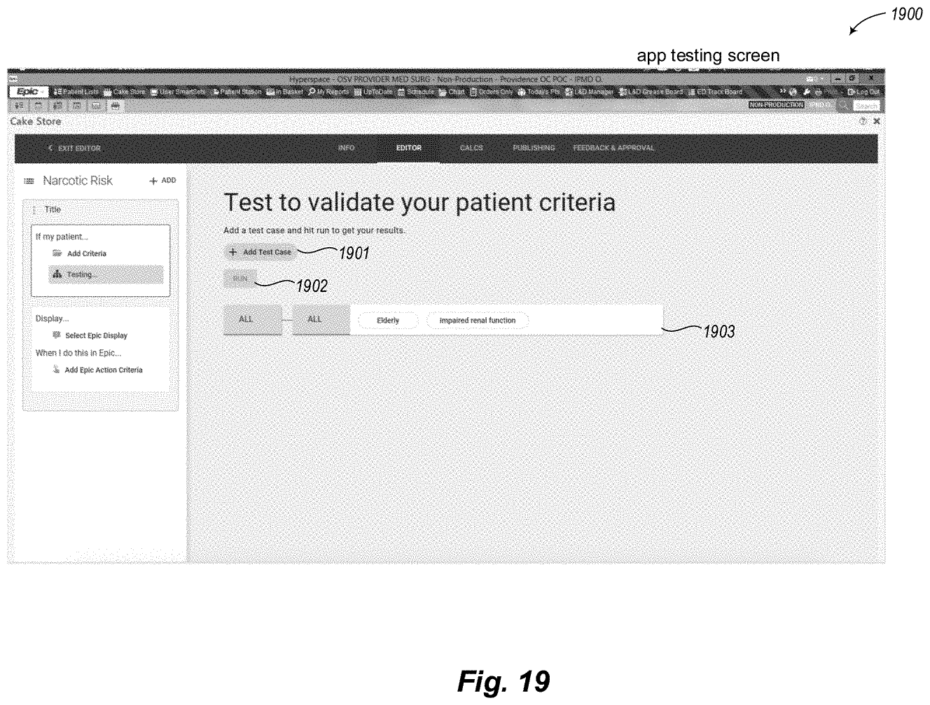

FIG. 19 is a display diagram showing a sample app testing screen presented by the facility in some embodiments. The app testing screen 1900 includes an add test button 1901, a run test button 1902, and a rule display 1903. The add test button 1901 allows a provider to input patient information to test if the app will select a patient with similar information. In some embodiments, a provider manually inputs patient information when adding a test case. In some embodiments, the facility obtains a list of patients from an EHR system and the provider selects one or more test cases from the list of patients. When the run test button 1902 is activated, the facility indicates to the provider which of the test cases were included based on the patient inclusion criteria. The rule display 1903 displays the patient inclusion rule as defined by the provider in act 1003. Here, the rule display 1903 indicates that the rule applies to elderly patients or patients with impaired renal function, so only test cases including patient data indicating that the patient is elderly or has impaired renal function are included when the rule is applied. In some embodiments, after validating the rule, the facility provides a suggestion to the provider when creating the rule, such as suggesting that the rule is too specific, not specific enough, includes too many patients, etc. In some embodiments, the facility provides a textual suggestion to the provider, such as, for example, “2 of 7 patients met this criteria.” In some embodiments, the facility provides a graphical suggestion to the provider by highlighting overly-specific rule components in display 1903, such as, for example, tinting the “Elderly” rule component if no test cases met the “Elderly” criterion.

Returning to FIG. 10, in act 1005, the facility receives user input specifying an action. In some embodiments, the facility performs act 1005 by utilizing an app action definition screen.

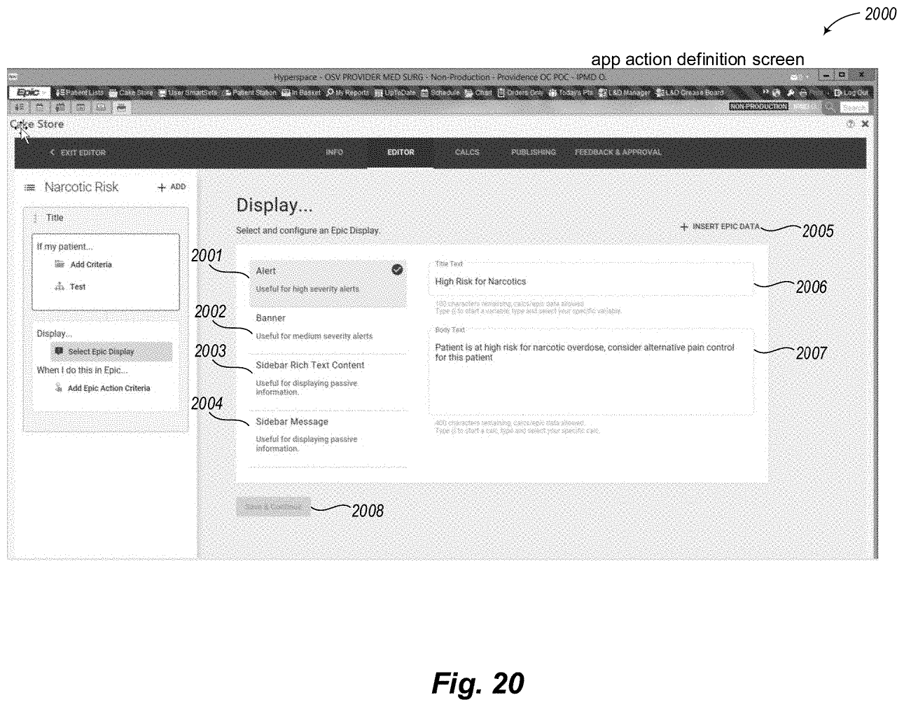

FIG. 20 is a display diagram showing a sample app action definition screen presented by the facility in some embodiments. The app action definition screen 2000 includes an alert definition tab 2001, a banner definition tab 2002, a sidebar rich text definition tab 2003, a side bar message tab 2004, an insert EHR data button 2005, and a save button 2008. Each of the tabs 2001-2004 correspond to a different type of action the facility can perform, such as displaying an alert, displaying a banner, displaying rich text content on a sidebar, or displaying a simple message on a sidebar. In some embodiments, the facility allows a provider to add other types of actions such as by sending direct messages, playing audio warnings, etc. After activating one of the tabs 2001-2004, the facility displays user interface elements the provider can use to input data corresponding to the action to be taken by the facility. For example, when the alert definition tab 2001 has been activated, the facility displays an alert title text box 2006 and an alert content text box 2007. The alert title text box 2006 allows a provider to input a title for the alert. The alert content text box 2007 allows a provider to input a message to display to a user of the app. The insert EHR data button 2005 allows a provider to indicate to the facility that data from an EHR system should be included in the action. For example, a provider could indicate to the facility that the patient's previous lab results, health history, medications, etc. are displayed with the action taken by the app, such as in an alert, banner, etc. When the save button 2008 is activated, the facility prompts the provider for an action trigger. In some embodiments, the facility prompts the provider for an action trigger by displaying an app action trigger definition screen.

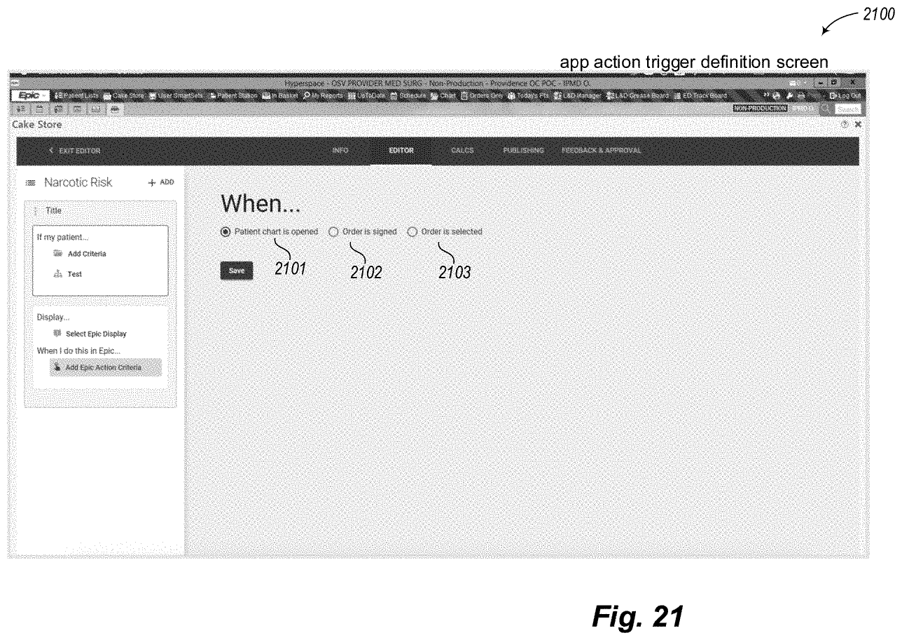

FIG. 21 is a display diagram showing a sample app action trigger definition screen presented by the facility in some embodiments. The app action trigger definition screen 2100 includes a patient chart radio button 2101, an order signed radio button 2102, and an order selected radio button 2103. Selecting the patient chart radio button 2101 indicates to the facility that the app should take an action when the patient chart is opened. Likewise, the order signed radio button 2102 indicates to the facility that the app should take an action when a provider signs an order, and the order selected radio button 2103 indicates the app should take an action when the provider selects an order. In some embodiments, the app action trigger definition screen 2100 allows the provider to select other action triggers, such as when new patient data is received by the facility, when the provider enters new patient information, when a patient sets a new appointment, etc. In some embodiments, the provider can also add parameters to the action trigger, for example, if the action trigger is an order, a provider can also include parameters indicating that only orders for a certain type of medication or treatment, such as narcotic medication, should activate the action trigger. In some embodiments, the action trigger can include ordering treatment or medication, ordering treatment or medication of a certain type, opening the patient chart, identifying new treatment methods for a patient, such as qualifying for a research study, new drug, newly discovered treatment, etc., receiving new EHR data related to the patient, receiving patient data from a non-EHR data source, receiving data related to other patients, etc.

Returning to FIG. 10, in act 1006, the facility receives user input specifying if the app should be beta tested. In some embodiments, the facility performs act 1006 by utilizing an app beta testing screen.

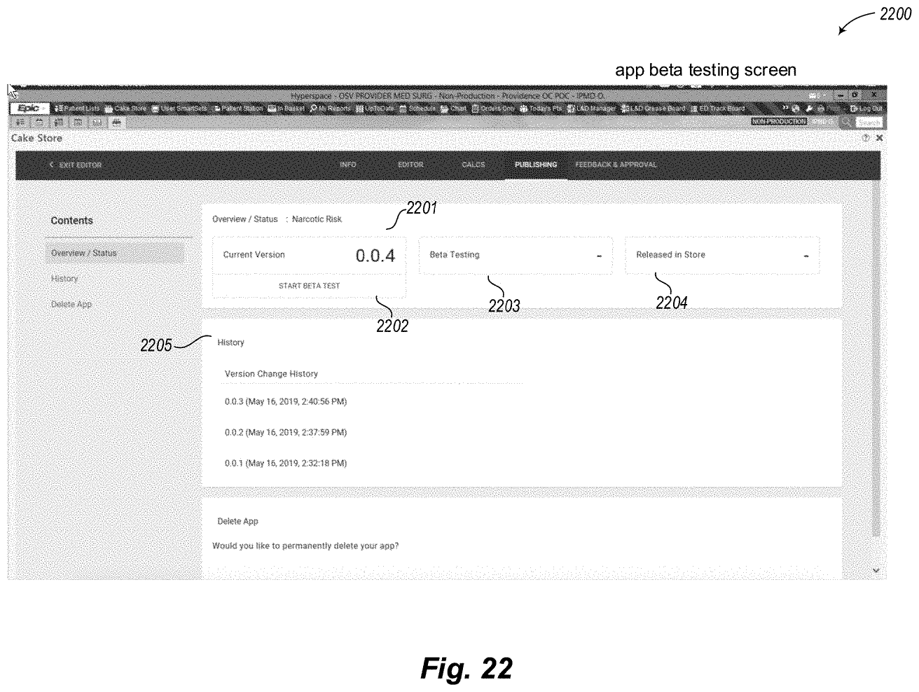

FIG. 22 is a display diagram showing a sample app beta testing screen presented by the facility in some embodiments. The app beta testing screen 2200 includes a current version indicator 2201, a start beta test button 2202, a testing status indicator 2203, a release indicator 2204, and a history report 2205. The current version indicator 2201 indicates the current version of the app. By activating the start beta test button 2202, the provider can make the app available to a certain number of other providers chosen to test the app before making it available to a greater number of providers in the healthcare system. In some embodiments, the facility allows providers who are beta testing an app to give feedback in the form of reviews or recommendations to improve the app. The testing status indicator 2203 indicates the testing stage the app is currently in, such as in development, beta testing, testing completed, etc. The release indicator 2204 indicates whether the app is released in an app store, unreleased, released for a certain group or number of providers, etc. The history report 2205 includes a summary of the various versions of the app.

Returning to FIG. 10, in act 1007, the facility utilizes the user input to create an app definition for an app that takes the specified action for patients selected by the patient inclusion criteria. In act 1008, the facility stores the created app in an app definition table. In some embodiments, when performing the process described in FIG. 10, the facility receives user input from a computing device with access to the app server. In some embodiments, when performing the process described in FIG. 10, the facility receives user input at a provider terminal. In some embodiments, when performing act 1008 the facility utilizes an app definition table. After act 1008, this process concludes.

FIG. 23 is a table diagram showing sample contents of an app definition table 2300 used by the facility in some embodiments. The app definition table 2300 contains rows, such as rows 2301-2303, each corresponding to a different app. Each row is divided into the following columns: an App ID column 2320, a Rule column 2321, an Owner column 2322, a Test column 2323, an Action column 2324, and a Name column 2325. The App ID column 2320 stores a unique identifier used to identify an app from other apps. In some embodiments, the facility automatically generates the identifier stored in the App ID column. The Rule column 2321 stores the patient inclusion rule used by the app. In some embodiments, the rule includes comparison operators and logical operators, such as, for example, the rule defined in row 2301 which includes patients who are at least 70 years old, have a BMI of at least 30, or have a GFR lab result of less than 60. In some embodiments, the rule is defined to include patients whose medical records include certain terms or phrases, such as the rule defined in row 2303 which includes patients whose medical records include the term “Seizure Risk.”

The Owner column 2322 indicates the owner or creator of the application. In some embodiments, the owner of the application is not the creator of the application. The Test column 2323 indicates the current testing status of the application. The Action column 2324 indicates when and what action the app takes when the facility has determined if a patient is included in the patient inclusion criteria defined by the rule stored in the rule column 2321. For example, the action defined in row 2301 instructs the facility to display an alert message when narcotic medication is ordered. In some embodiments, the facility uses two columns for the Action column 2324, one storing the action for the app to perform, and one storing the action trigger (when to perform the action). The Name column 2325 indicates the name displayed by the facility when displaying information related to the app, when the app is displayed in the app store, etc. In some embodiments, the app definition table 2300 includes columns for additional data such as a version number, a changelog, an external data source for retrieving data outside of the EHR system, keywords used to identify the app, etc.

While FIG. 23 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc.

In some embodiments, when the facility performs the processes described in FIGS. 5 and 10, the facility provides a suggestion to the provider when creating the rule, such as suggesting that the rule is too specific or not specific enough. In some embodiments, when the facility performs the processes described in FIGS. 5 and 10, the facility pre-loads criteria based on a patient's chart, for example: if a patient is 65 years old, then when the provider adds criteria concerning age, the age in years will be set to 65 years old automatically, and the provider can then change that parameter to include or exclude other potential patients.

In some embodiments, when the facility performs the processes described in FIGS. 5 and 10, the facility utilizes machine learning to automatically generate rules relevant to a patient's chart. In some embodiments, when the facility performs the processes described in FIGS. 5 and 10, the facility utilizes machine learning to predict if a patient's chart is atypical, and alerts the provider to that fact before the provider creates the app. In some embodiments, the facility uses machine learning to suggest rules based on historical patient information, physiologic reference data (e.g., lab value ranges, vital signs ranges, etc.), previously-built rules, and current app metadata (e.g., app name, description, etc.) to identify patient characteristics that the provider may want to consider for a rule.

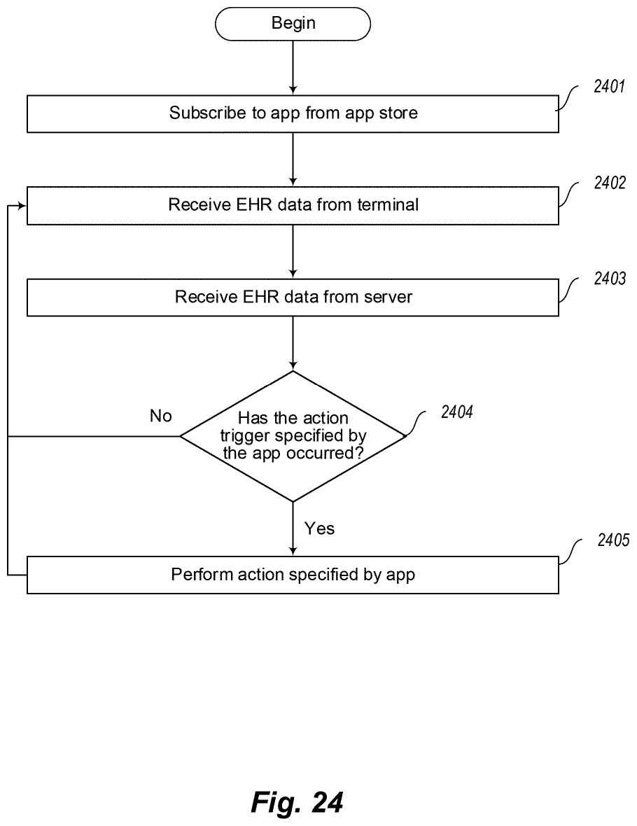

FIG. 24 is a process to subscribe to and use an app used by the facility in some embodiments. In act 2401, the provider subscribes to an app displayed in an app store. In some embodiments, the provider accesses the app store and subscribes to the app form the provider terminal. In some embodiments, the facility automatically subscribes a provider to the app. In some embodiments, the provider accesses the app store and subscribes to the app from a computing device other than the provider terminal, such as a personal computer, a laptop, or another computing device in the healthcare computer network. In some embodiments, the facility utilizes an app store table 2500 to manage the apps available to a subscriber. In some embodiments, the facility utilizes an app store screen 2600 to perform act 2401.

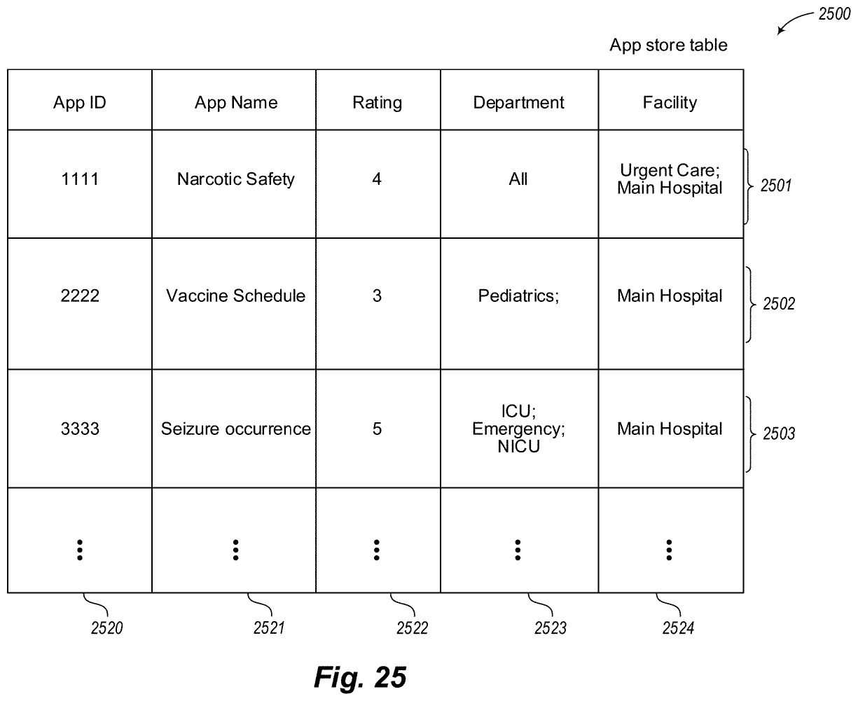

FIG. 25 is a table diagram showing sample contents an app store table 2500 used by the facility in some embodiments. The app store table 2500 contains rows, such as rows 2501-2503, each corresponding to a different app. Each row is divided into the following columns: an App ID column 2520, an App Name column 2521, a Subscribed by column 2522, a Rating column 2523, a Department column 2524, and a Facility column 2525. The facility stores a unique identifier in the App ID column 2520 which corresponds to the App ID column 2320. Likewise, the facility stores a name in the App Name column 2521 which corresponds to the Name column 2325. The Subscribed by column 2522 indicates which providers have subscribed to the app from the app store. The Rating column 2523 indicates an average rating based on the ratings of the app provided by the providers. The Department column 2524 indicates which departments are able to subscribe to the app. For example, providers in any department can subscribe to the Narcotic Safety app in row 2501; however, only providers in the Pediatrics department can subscribe to the Vaccine Schedule app in row 2502. The Facility column 2525 indicates whether providers in a certain healthcare facility is able to subscribe to the app. As shown in FIG. 25, providers in the Urgent Care facility and Main Hospital can subscribe to the Narcotic Safety app in row 2501; however, only providers in the Main Hospital can subscribe to the Vaccine Schedule app in row 2502. In some embodiments, the app store table includes columns indicating reviews of the app, recommendations for improvements to the app, a threshold number of subscribers needed to make the app available to any provider, etc.

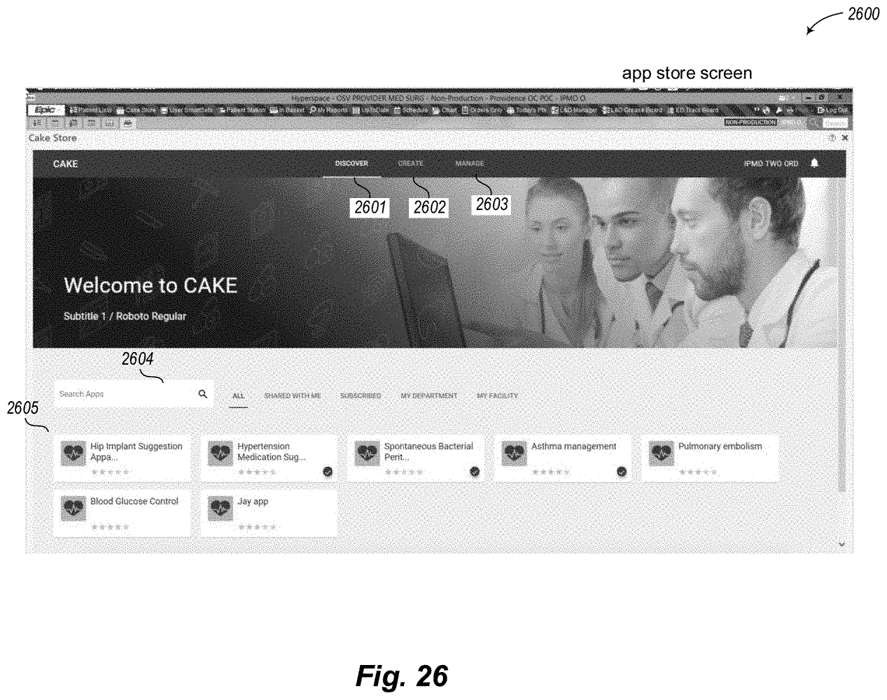

FIG. 26 is a display diagram showing a sample app store screen 2600 presented by the facility in some embodiments. The app store screen 2600 includes a discover tab 2601, a create tab 2602, and a manage tab 2603. In some embodiments, the facility displays the app store screen 2600 inside of an existing EHR system by displaying the app store screen 2600 within an iframe displayed by the EHR system. The discover tab 2601 displays apps available on the app store for a provider to subscribe to, including a search bar 2604 and an app list 2605. The search bar 2604 allows a provider to search for apps, such as through a keyword search, searching for apps which take specific actions, searching for apps which operate on a specified type of medical data, searching for apps with certain tags, etc. The facility displays apps in an app list 2605 by displaying the name, rating, and whether the provider has already subscribed to the app. In some embodiments, the facility populates the app list with apps which match search terms entered in the search bar 2604. When a provider activates the create tab 2603, the facility allows the provider to create a new app using the processes described in FIG. 5 and FIG. 10. When a provider activates the manage tab 2603, the facility displays a list of apps for which the provider is listed as an owner, and allows the provider to manage app testing, make changes to the app, release the app to more providers on the network, etc. In some embodiments, the facility utilizes machine learning to recommend an app to a provider while the provider is browsing the app store. In some embodiments, the facility uses machine learning to suggest apps based on a provider's characteristics, such as medical specialty, location, department, prescribing habits, etc.

Returning to FIG. 24, in act 2402 the facility receives EHR data from the provider terminal. In some embodiments, the terminal receives EHR data from the EHR system, and the facility receives the EHR data by accessing data already present on the terminal. In some embodiments, the facility receives the EHR data directly from the EHR system. In some embodiments, the facility receives data from a source outside the EHR system. In act 2403, the facility receives EHR data from the app server. In some embodiments, act 2403 is not performed. In some embodiments, in acts 2402 and/or 2403, the facility receives the EHR data asynchronously. In some embodiments, in acts 2402 and/or 2403, the facility requests EHR data from the EHR system using tools designed to exchange data between EHR systems, such as FHIR or Redox. In some embodiments, in acts 2402 and/or 2403, the facility requests EHR data from the EHR system by using HL7, HTTPS, REST, SOAP, or other communication protocols.

In some embodiments, the facility utilizes a “patient almanac” to gather and evaluate EHR data. In some embodiments, the facility uses the patient almanac to asynchronously obtain patient data when needed, caches the data, and then updates the data when new data is available, and then uses the data stored in the patient almanac instead of accessing the EHR system when data in the patient almanac is available. In some embodiments, the facility obtains patient data by accessing data already stored on the computing device operating the app. In some embodiments, the facility stores the data obtained by accessing data already stored on the computing device operating the app in the patient almanac.

In act 2404, the facility determines if the action trigger specified by the app has occurred. If the action trigger has not occurred, the process returns to act 2402. If the action trigger has occurred, the process continues to act 2405. In act 2405, the facility performs the action specified by the app, and then the process returns to act 2402. In some embodiments, the action taken by the facility is to display an app alert screen. In some embodiments, the action taken by the facility is to display an app banner notification screen. In some embodiments, the action taken by the facility is to display an app push notification screen. After act 2405, the process concludes.

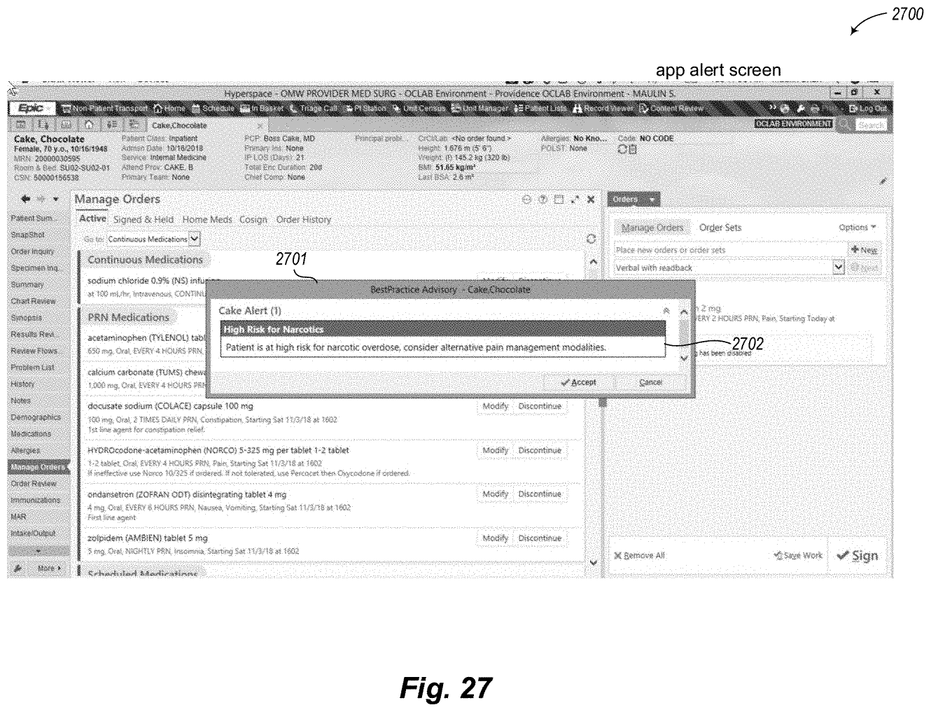

FIG. 27 is a display diagram showing a sample app alert screen used by the facility in some embodiments. The app alert screen 2700 includes an alert 2701 and an alert message 2702. The alert 2701 is a pop-up alert and is displayed when the facility detects an action trigger has occurred, such as ordering a narcotic medication. The alert message 2702 is a message specified by the provider which the facility displays within the alert 2701.

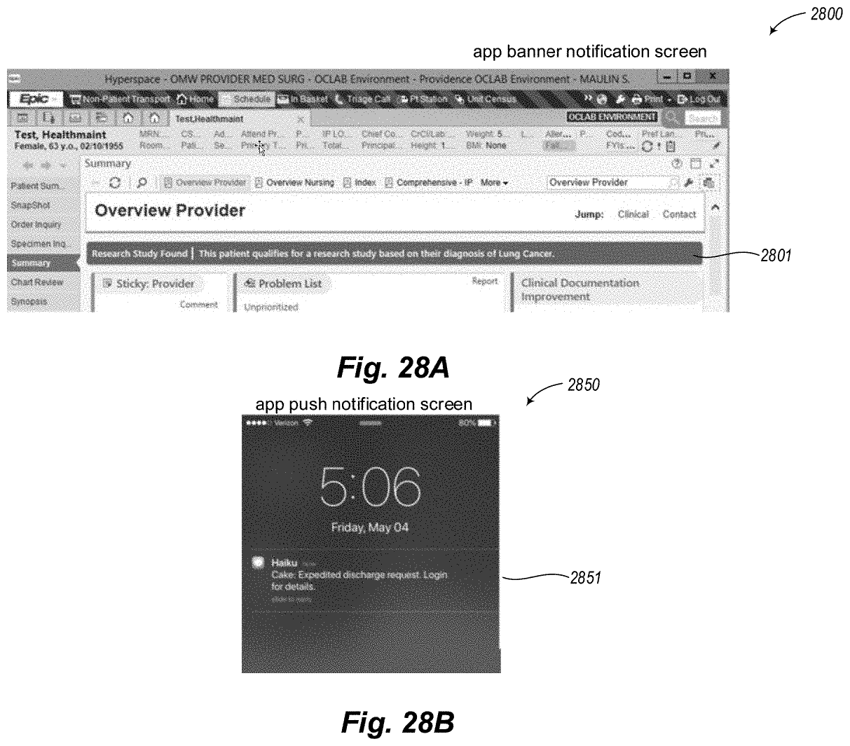

FIG. 28A is a display diagram showing a sample app banner notification screen used by the facility in some embodiments. The app banner notification screen 2800 includes a banner notification 2801. The banner notification 2801 is displayed at the top of the screen displayed by the provider terminal, and includes the message and title specified by the provider when the provider created the app.

FIG. 28B is a display diagram showing a sample app push notification screen, used by the facility in some embodiments. The app push notification screen 2850 includes a notification 2851. The app push notification screen 2850 can be displayed by a computing device within the healthcare computer network, or by a computing device outside of the healthcare computer network, such as a cellular telephone, personal computer, laptop computer, tablet computer, personal digital assistant, etc. The notification 2851 can include the message identified by the provider. In some embodiments, the notification 2851 prompts the provider to enter login information to view the message.

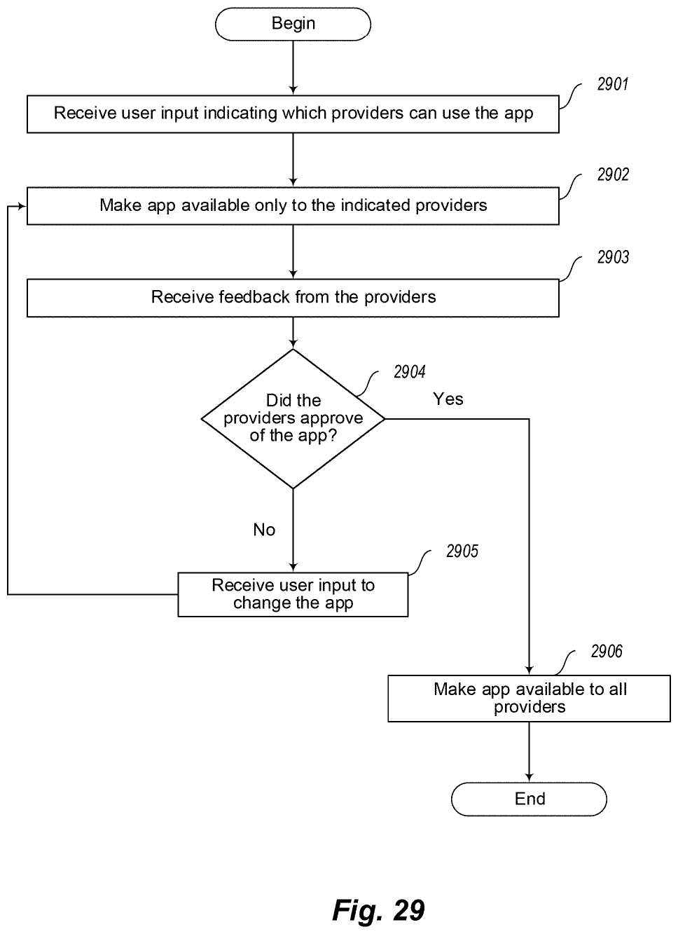

FIG. 29 is a process to approve apps used by the facility in some embodiments. In act 2901, the facility receives user input indicating which providers can subscribe to and use the app. In some embodiments, in performing act 2901, the facility receives user input identifying specific providers. In some embodiments, in performing act 2901, the facility receives user input identifying a group or groups of providers. In act 2902, the facility makes the app available only to the providers identified in act 2901. In some embodiments, as part of performing act 2901 or 2902, the facility receives user input indicating a threshold number of providers needed to approve of the app. In act 2903, the facility receives feedback from the providers. In some embodiments, the facility obtains feedback and sends it directly to the owner or creator of the app. In some embodiments, the facility allows providers to provide feedback within the app store in the form of user reviews.

In act 2904, if a predetermined number of providers has approved of the app the process continues to act 2906, otherwise the process continues to act 2905. In act 2905, the facility receives user input from the owner, or creator, of the app to alter or edit the app, and then returns to act 2902.

In act 2906, the facility makes the app available to all providers. In some embodiments, the facility makes the app available only to providers in a certain department or facility within the healthcare network. In some embodiments, the facility utilizes an app deployment table 3000 in performing the process to approve apps. After act 2906, the process concludes.

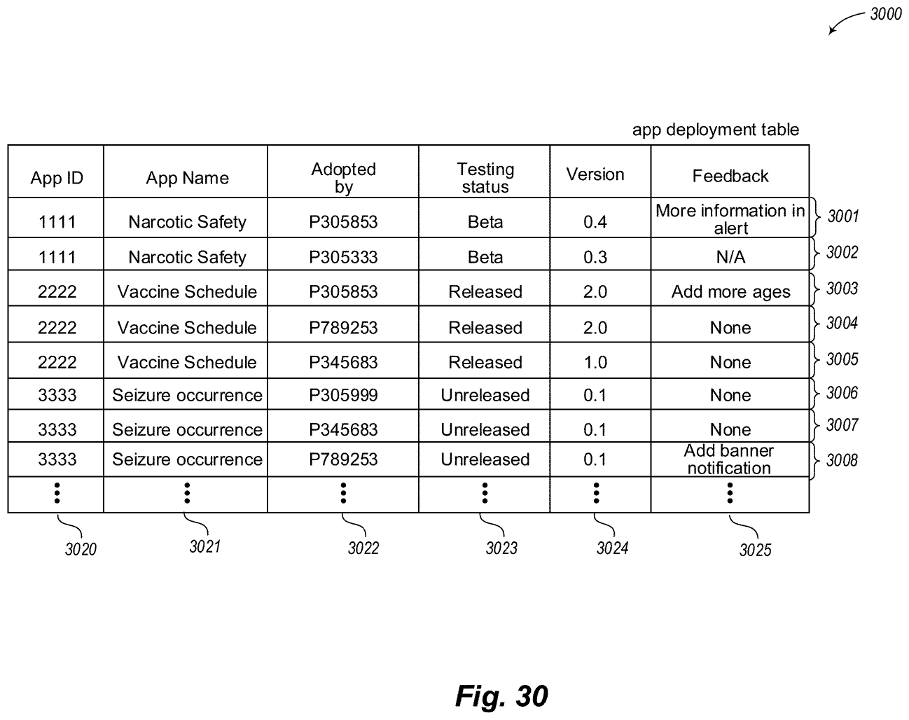

FIG. 30 is a table diagram showing sample contents an app deployment table 3000 used by the facility in some embodiments. The app deployment table 3000 contains rows, such as rows 3001-3008, each corresponding to a different app. Each row is divided into the following columns: an App ID column 3020, an App Name column 3021, an Adopted by column 3022, a Testing status column 3023, a Version column 3024, and a Feedback column 3025. The values of the App ID column 3020 and App name column 3021 correspond to the values of the App ID column 2320 and Name column 2325 of the app definition table 2300, and are used to identify the app. The Adopted by column 3022 lists the provider which has adopted, or subscribed to, the app. The Testing status column 3023 indicates whether the app is being beta tested, is released, is unreleased, etc. The Version column 3024 indicates the version of the app that the provider identified in the Adopted by column 3022 is currently using. The Feedback column 3025 indicates any feedback provided by the provider identified in the Adopted by column 3022.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. One or more instances of computer-readable media collectively having contents configured to cause a computing device to perform a method for accessing patient data in an EHR system, the method comprising:
receiving, via a first user interface element, user input specifying an action trigger;
receiving, via a second user interface element, distinct from the first user interface element, user input specifying an action; and
in response to receiving the user input, creating an application that:
compares patient data stored in the EHR system to patient inclusion criteria;
determines whether the patient data stored in the EHR system is specified by the patient inclusion criteria;
detects if the action trigger has occurred; and
causes the computing device to perform the action when the action trigger occurs based on the determination of whether the patient data stored in the EHR system is satisfies the patient inclusion criteria.

2. The one or more instances of computer-readable media of claim 1, the method further comprising:
receiving, via a third user interface element, distinct from the first user interface element and the second user interface element, user input specifying the patient inclusion criteria.

3. The one or more instances of computer-readable media of claim 1, wherein the created application is configured to execute for a distinguished patient when an EHR record for the patient is accessed at the patient terminal.

4. The one or more instances of computer-readable media of claim 1, wherein created application is configured to execute for a distinguished patient when an EHR record of the patient is updated.

5. The one or more instances of computer-readable media of claim 1, wherein the action specified by the user input received via the second user interface element is a message to be transmitted to a provider computer device.

6. The one or more instances of computer-readable media of claim 1, wherein the action specified by the user input received via the second user interface element is a message to be displayed within an alert dialog.

7. The one or more instances of computer-readable media of claim 1, wherein the method further comprises:
uploading the application to a directory of applications designed to distribute applications within an EHR system.

8. The one or more instances of computer-readable media of claim 1, wherein the method further comprises:
receiving, via a third user interface element, distinct from the first user interface element and the second user interface element, user input specifying an additional data source, wherein the created application is configured to retrieve additional EHR data from the additional data source.

9. The one or more instances of computer-readable media of claim 1, wherein the method further comprises:
in response to receiving user input via the first or second user interface elements, displaying one or more messages to the user, wherein the messages include suggestions to improve the functionality of the application.

10. A system for accessing patient data in an EHR system, the system comprising:
a server configured to access an EHR system, the server being further configured to store applications;
a computing device in communication with the server, the computing device being configured to display EHR data, the EHR data including patient data;
the computing device being further configured to:
receive user input specifying patient inclusion criteria and an action while the computing device displays the patient data; and
create an application, the application being configured to:
compare patient data displayed by the computing device to the received patient inclusion criteria;
determine whether the patient data displayed by the computing device is satisfies the patient inclusion criteria;
cause the computing device to take the action based on the determination of whether the patient data displayed by the computing device satisfies the patient inclusion criteria.

11. The system of claim 10, further comprising:
the computing device being further configured to:
receive user input specifying an action trigger while the computing device displays the patient data; and
cause the application to be further configured to:
determine whether the action trigger has occurred; and
cause the computing device to take the action based on the determination of whether the action trigger has occurred and the determination of whether the patient data displayed by the computing device satisfies the patient inclusion criteria.

12. The system of claim 11, wherein the action specified by the user input is to transmit a message to a provider computer device.

13. The system of claim 11, wherein the action specified by the user input is to display an alert dialog containing a message.

14. The system of claim 10, further comprising:
an application distribution server, the application distribution server being configured to:
receive applications from the computing device; and
transmit applications to other computing devices.

15. The system of claim 10, further comprising:
the computing device being further configured to:
receive user input specifying a data source; and
cause the application to be further configured to:
receive additional patient data from the data source; and
alter the patient data to include the additional patient data.

16. The system of claim 10, further comprising:
the computing device being further configured to display messages to the user in response to receiving the user input, the messages including suggestions to improve the application.

17. The system of claim 10, further comprising:
the computing device being further configured to execute multiple applications, such that each application is able to take an action when the computing device determines the patient inclusion criteria is satisfied.

18. The system of claim 10, further comprising:
the computing device being further configured to allow multiple users to use the application.

19. The system of claim 10, further comprising:
an approved provider list, the approved provider list including a list of users allowed to use the application; and
the computing device being further configured to allow only users included in the approved provider list to use the application.

20. One or more storage devices collectively storing an application data structure, the data structure comprising:
information specifying patient inclusion criteria;
information specifying an action trigger; and
information specifying an action,
such that the contents of the data structure are usable to operate an application that causes a computing device to perform the action in response to patient data being uploaded to the computing device and the action trigger being satisfied.

21. The one or more storage devices of claim 20, wherein the data structure further comprises:
information specifying a data source,
such that the data source is usable to provide additional patient data.

22. The one or more storage devices of claim 20, wherein the data structure further comprises:
information specifying a list of approved providers,
such that the application created by using the contents of the data structure is usable only to providers included in the list of approved providers.

23. The one or more storage devices of claim 20, wherein the data structure further comprises:
information specifying a message,
such that the application created by using the contents of the data structure displays the message when the computing device takes the action.

24. The one or more storage devices of claim 20, wherein the data structure further comprises:
information specifying additional patient inclusion criteria,
such that the application created by using the contents of the data structure causes the computing device to retrieve additional patient data from patients other than the patient satisfies the patient data that satisfy the additional patient inclusion criteria.

25. The one or more storage devices of claim 22, wherein the data structure further comprises:
information specifying whether a providers included in the approved provider list are allowed to access applications available for a limited release; and
information specifying whether the application is available for a limited release, such that the application created by using the contents of the data is usable only to providers included in the list of approved providers allowed to access applications available for a limited release.

26-47. (canceled)
